(19) United States
(12) Patent Application Publication
(10) Pub. No.: US 2015/0017093 A1
(43) Pub. Date: Jan. 15, 2015

(54) RADIOLABELED BILE ACIDS AND BILE ACID DERIVATIVES

(71) Applicants: Aarhus Universitet, Arhus C (DK); Region Midtjylland, Viborg (DK)

(72) Inventors: Susanne Keiding, Hojbjerg (DK); Peter Ott, Hojbjerg (DK); Michael Sorensen, Arhus N (DK); Kim Frisch, Skodstrup (DK); Alan Hofmann, La Jolla, CA (US)

(73) Assignees: Aarhus Universitet, Arhus C (DK); Region Midtjylland, Viborg (DK)

(21) Appl. No.: 14/375,653

(22) PCT Filed: Jan. 29, 2013
(86) PCT No.: PCT/EP2013/051635

§ 371 Date: Jul. 30, 2014

Related U.S. Application Data
(60) Provisional application No. 61/649,504, filed on May 21, 2012.
(60) Provisional application No. 61/594,430, filed on Feb. 3, 2012.

(51) Int. Cl. A61K 51/04; C07J 41/00

(52) U.S. Cl. CPC A61K 51/0493; C07J 41/0061
USPC 424/1.45; 552/554

(57) ABSTRACT

The present invention relates in one aspect to radiolabeled compounds comprising the structure of Formula 1. The radiolabeled compounds are preferably bile acids or bile acid derivatives. Further aspects of the invention relates to use of the compounds comprising the structure of Formula 1 in imaging methods such as for example PET, imaging method using a compound comprising the structure of Formula 1, administering said compound to an individual and making a radiographic image of a region of interest from said individual.

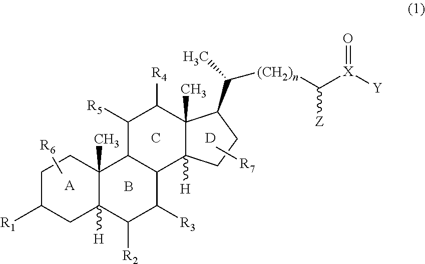

### FIELD OF INVENTION

The present invention relates in one aspect to radiolabeled compounds comprising the structure of Formula 1. The radiolabeled compounds are preferably bile acids or bile acid derivatives. Further aspects of the invention relates to use of the compounds comprising the structure of Formula 1 in imaging methods such as for example PET, imaging method using a compound comprising the structure of Formula 1, administering said compound to an individual and making a radiographic image of a region of interest from said individual.

### BACKGROUND OF INVENTION

The hepatic uptake and subsequent biliary excretion of organic solutes is a key liver function in which bile acids play an important role. Bile acids are synthetized in the liver cells (hepatocytes) and excreted into the bile ducts. Bile acids stimulate bile flow and in the small intestines, they facilitate uptake of lipophilic substances and the secretion of bile acids serves as a way to eliminate cholesterol and toxic substances. After intestinal reabsorption the bile acids enter the portal vein, which drains the intestines. The portal vein blood enters the liver sinusoids and bile acids are transported from the blood to the hepatocytes. This enterohepatic circulation of the bile acids amounts up to 15 circulations per bile acid per day. Both efficient hepatocellular uptake of bile acids from blood and excretion of bile acids into the bile ducts are hence important processes. Impairment of these processes lead to cholestasis, which is a feature of a large variety of inherited and acquired liver diseases in which accumulation of bile acids in the hepatocytes is thought to be an important pathogenetic factor in liver injury. In this situation a non-invasive method that quantifies the transhepatic transport of bile acids would be of considerable interest given its potential to improve our understanding of normal physiology, pathophysiology, and the hepatic effects or side effects of new and known drugs. To date, such a method is not available. Dynamic planar ^{99m}Tc-mebrofenin scintigraphy, which has been used to assess biliary excretion does not provide the necessary quantitative information.

Most cancer cells have an increased glucose metabolism and positron emission tomography (PET) using the glucose analogue [¹⁸F] 2-deoxy-2-fluoro-D-glucose (¹⁸F-FDG) is widely used for diagnosis of cancer.

The use of ¹⁸F-FDG for human hepatocellular carcinoma (HCC) is problematic because of similarities concerning the glucose metabolism between normal hepatocytes and tumour cells derived from hepatocytes. ¹¹C-Acetate has been proposed as an alternative radiotracer for detecting HCC lesions not revealed by ¹⁸F-FDG because HCC cells with low glycolysis show increased ¹¹C-acetate uptake when compared with surrounding liver tissue (Ho C L et al., J Nucl Med 2003; 44:213-21). ¹⁸F-Fluoroacetate (FAC), an analogue of acetate, is metabolized to fluoroacetyl-CoA and then fluorocitrate, which cannot be further metabolized to CO₂ and water, thus trapped in the cell in proportion to oxidative metabolism.

The use of ¹¹C-choline in PET has been reported for the detection of tumours in the brain and prostate, even for tumour recurrence (Fallanca F et al., Q J Nucl Med Mol Imaging 2009; 53:417-21). It has been found that ¹¹C-choline PET had a higher overall detection rate for HCC lesions than ¹⁸F-FDG or ¹¹C-acetate (Salem N et al., Q J Nucl Med Mol Imaging, 2009; 53:144-56).

Recently the use of [¹⁸F] 2-deoxy-2-fluoro-D-galactose (¹⁸F-FDGal) has been found promising for diagnosis of HCC (Sørensen M et al, Eur J Nucl Med Mol Imaging 2011; 38:1723-31).

The drug telmisartan is eliminated via biliary excretion and [¹¹C]telmisartan has been used in PET imaging to analyze the hepatobiliary transport of this compound in rats. Data analysis revealed that [¹¹C]telmisartan was taken up into the liver as rapidly as the hepatic blood flow rate and that the ¹¹C radio metabolite was subsequently excreted into the bile (Takashima et al., Mol. Pharmaceutics, 2011, 8 (5):1789-1798).

N—¹¹C-acetyl-leukotriene E₄ has been investigated as a PET tracer for assessment of the ratio of biliary to renal elimination of leukotrienes (Guhlmann et al., Hepatology 1995; 21:1568-1575). However, hepatorenal syndrome is associated with increased renal excretion of cysteinyl leukotrienes and thus, N—¹¹C-acetyl-leukotriene will probably not be a suitable tracer for biliary excretion in liver diseases.

¹¹C-labelled (15R)-16-m-tolyl-17,18,19,20-tetranorisocarbacyclin methyl ester ((15R)-¹¹C—TlC-Me) has been investigated for quantification of transport via OATP1B1 and OATP1B3 by PET in humans with metabolites excreted into bile possibly via MRP2 (Takashima et al., J Pharmacol Exp Ther 2010; 335:314-323 and J Nucl Med 2012; 53:741-748). However, functional discrimination between OATP1B1 and OATP1B3 by (15R)-¹¹C—TlC-Me PET is difficult if not impossible.

The high spatial and temporal resolutions of contemporary PET suggest that PET/CT could be a suitable methodology to study the hepatic handling of bile acids but so far no specific bile acid tracers have been developed. The ideal tracer should have biochemical similarities with common bile acids, a high first pass hepatic extraction, and be excreted in high concentrations in bile. In addition, the transhepatic transport of the ideal tracer should be mediated by the same transporter proteins that facilitate the transport of the common bile acid conjugate cholyltaurine, i.e. the sodium-taurocholate cotransporting polypeptide (NTCP) and organic anion transporting polypeptides (OATPs) from blood to hepatocytes and the bile salt export pump (BSEP) from hepatocyte to bile (Alrefai W, Gill R. Bile acid transporters: Structure, function, regulation and pathophysiological implications. Pharm Res. 2007; 24:1803-1823.). Moreover, kinetic analysis is simpler if the tracer is not metabolized.

Accordingly, there is a need for radiolabeled compound having biochemical similarities with common bile acids, which can be used as tracers in imaging methods such as for example PET.

### SUMMARY OF INVENTION

The objective of the present invention is to provide compounds that are useful to study the hepatic and intestinal handling of bile acids.

Accordingly, a first aspect of the present invention relates to radiolabeled compound comprising the structure of Formula 1:

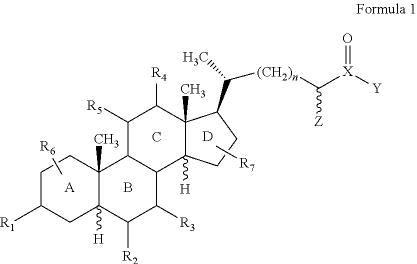

or a salt and/or hydrate thereof;
wherein:
   said compound comprises a steroid structure (ABCD) and at least one radioactive isotope selected from the group consisting of ¹¹C and ¹⁸F
   n is 0, 1, 2 or 3
   X is C or ¹¹C
   Z is H or —CH₃
   Y is selected from the group consisting of OH, OR₈, C₁₋₆-alk(en/yn)yl, NR₉R₁₀
   R₁, R₂, R₃, R₄, R₅, R₆ and R₇ are individually selected from the group consisting of H, OH, C₁₋₆-alk(en/yn)yl, aryl, halo-C₁₋₆-alk(en/yn)yl, C₃₋₈-cycloalk(en)yl, halo-C₃₋₈-cycloalk(en)yl, and hydroxy-C₁₋₆-alk(en/yn)yl, cyano, halogen, oxo, OSO₂OH, —CF₃, and NR₁₁R₁₂
   R₈ is selected from the group consisting of C₁₋₆-alk(en/yn)yl, aryl and C₃₋₈-cycloalk(en)yl, halo-C₁₋₆-alk(en/yn)yl, halo-C₃₋₈-cycloalk(en)yl
   R₉ is selected from the group consisting of H, C₁₋₆-alk(en/yn)yl, —COOH, —CHO, —CH₂COOH, —CH₂COOC₁₋₄alkyl, —CH₂SO₂OH, —CH₂CH₂COOH, —CH₂CH₂COOC₁₋₄alkyl, —CH₂CH₂SO₂OH, —(CH₂)₁₋₄N⁺R₁₃R₁₄R₁₅, —CH(CH₃)COOH, —CH((CH₂)₃NHC(NH)NH₂)COOH, —CH(CH₂CONH₂)COOH, CH(CH₂COOH)COOH, —CH(CH₂SH)COOH, —CH(CH₂CH₂CONH₂)COOH, —CH(CH₂CH₂COOH)COOH, —CH(CH₂C₃N₂H₃)COOH, —CH(CH(CH₃)CH₂CH₃)COOH, —CH((CH₂)₄NH₂)COOH, —CH((CH₂)₂SCH₃)COOH, —CH(CH₂C₆H₅)COOH, —CH(CH₂OH)COOH, —CH(CH(CH₃)OH)COOH, —CH(CH₂C₈H₆)COOH, —CH(CH₂C₆H₄OH)COOH, —CH(CH(CH₃)₂)COOH
   R₁₀ is selected from the group consisting of H, C₁₋₈-alk(en/yn)yl, ¹¹CH₃, —(CH₂)₁₋₈F, —(CH₂)₁₋₈¹⁸F, CH₂—C₃₋₈-cycloalk(en)yl and CH₂-halo-C₃₋₈-cycloalk(en)yl
   R₁₁ and R₁₂ are individually selected from the group consisting of H, C₁₋₆-alk(en/yn)yl, ¹¹CH₃, aryl, C₃₋₈-cycloalk(en)yl
   R₁₃, R₁₄ and R₁₅ are individually selected from the group consisting of C₁₋₈-alk(en/yn)yl.

The steroid structure (ABCD) may in one embodiment comprise one or more double bonds.

In a preferred embodiment R₁, R₃ and R₄ is H or OH. In a more preferred embodiment R₁ is OH. In a specific embodiment R₁, R₃ and R₄ is OH, preferably in an α-position.

In the compound of Formula 1 n is 0, 1, 2 or 3. Preferable n is 1.

In another preferred embodiment R₂, R₅, R₆ and R₇ is H.

It one embodiment Y is OH. In another preferred embodiment Y is NR₉R₁₀. Preferably R₉ is CH₂COOH.

Formula 2

In further embodiments, the invention relates to compounds or salts of Formula 1, such as compounds comprising the substructure of Formula 2:

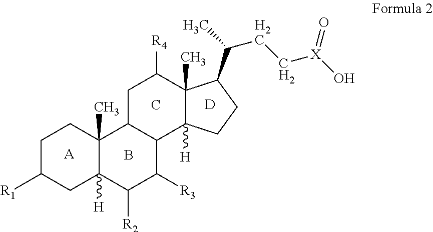

or a salt and/or hydrate thereof;
wherein:
   X is ¹¹C
   R₁, R₂, R₃ and R₄ are individually selected from the group consisting of H, OH, C₁₋₆-alk(en/yn)yl, aryl, halo-C₁₋₆-alk(en/yn)yl, C₃₋₈-cycloalk(en)yl, halo-C₃₋₈-cycloalk(en)yl, and hydroxy-C₁₋₆-alk(en/yn)yl, cyano, halogen, oxo, OSO₂OH, —CF₃, and NR₁₁R₁₂
   R₁₁ and R₁₂ are individually selected from the group consisting of H, C₁₋₆-alk(en/yn)yl, aryl, C₃₋₈-cycloalk(en)yl.

In one embodiment, wherein the compound of Formula 1 comprises the substructure of Formula 2, R₁ is OH. In a preferred embodiment R₁ and R₄ are OH. In a more preferred embodiment R₁, R₃ and R₄ are OH. Preferably R₁, R₃ and R₄ are OH in α-position. In a preferred embodiment R₂ is H.

In a particular preferred embodiment the compound of Formula 1 comprises the substructure of Formula 2, wherein
X is ¹¹C
R₁, R₃ and R₄ are OH
R₂ is H

It is preferred that R₁, R₃ and R₄ are OH in α-position.

Formula 3

In further embodiments, the invention relates to compounds or salts of Formula 1, such as compounds comprising the substructure of Formula 3:

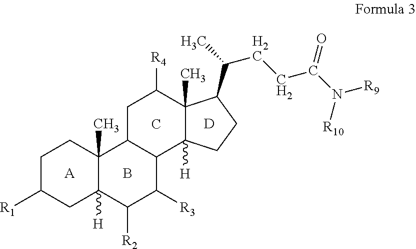

or a salt and/or hydrate thereof;

wherein:
R₁, R₂, R₃, and R₄ are individually selected from the group consisting of H, OH, C₁₋₆-alk(en/yn)yl, aryl, halo-C₁₋₆-alk(en/yn)yl, —C₃₋₈-cycloalk(en)yl, halo-C₃₋₈-cycloalk(en)yl, and hydroxy-C₁₋₆-alk(en/yn)yl, cyano, halogen, oxo, OSO₂OH, —CF₃, and NR₁₁R₁₂
R₉ is selected from the group consisting of H, —C₁₋₆-alk(en/yn)yl, —COOH, —CHO, —CH₂COOH, —CH₂COOC₁₋₄alkyl, —CH₂SO₂OH, —CH₂CH₂COOH, —CH₂CH₂COOC₁₋₄alkyl, —CH₂CH₂SO₂OH, —(CH₂)₁₋₄N⁺R₁₃R₁₄R₁₅
R₁₀ is selected from the group consisting of H, —C₁₋₈-alk(en/yn)yl, —¹¹CH₃, —(CH₂)₁₋₈F, —(CH₂)₁₋₈¹⁸F, —CH₂—C₃₋₈-cycloalk(en)yl, —CH₂— halo-C₃₋₈-cycloalk(en)yl
R₁₁ and R₁₂ are individually selected from the group consisting of H, C₁₋₆-alk(en/yn)yl, aryl, C₃₋₈-cycloalk(en)yl
R₁₃, R₁₄ and R₁₅ are individually selected from the group consisting of —C₁₋₈-alk(en/yn)yl.

In one embodiment, wherein the compound of Formula 1 comprises the substructure of Formula 3, R₁, R₂, R₃ and R₄ are individually selected from the group consisting of H and OH. In a preferred embodiment R₁, R₃ and R₄ are OH and R₂ is H. In a more preferred embodiment R₁, R₃ and R₄ are OH in α-position and R₂ is H.

In one embodiment, wherein the compound of Formula 1 comprises the substructure of Formula 3, R₉ is —CH₂COOH and R₁₀ is —CH₂¹⁸F. In a particular preferred embodiment, wherein the compound of Formula 1 comprises the substructure of Formula 3, R₉ is —CH₂COOH and R₁₀ is —¹¹CH₃.

In one embodiment, wherein the compound of Formula 1 comprises the substructure of Formula 3, R₉ is —CH₂CH₂SO₂OH and R₁₀ is —CH₂¹⁸F. In a more embodiment embodiment, wherein the compound of Formula 1 comprises the substructure of Formula 3, R₉ is —CH₂CH₂SO₂OH and R₁₀ is —¹¹CH₃.

In a particular preferred embodiment the compound of Formula 1 comprises the substructure of Formula 3, wherein
R₁, R₃ and R₄ are OH
R₂ is H
R₉ is —CH₂CH₂SO₂OH
R₁₀ is —CH₂¹⁸F

In a preferred embodiment R₁, R₃ and R₄ are OH in α-position. This compound is equivalent to ¹⁸F-cholyltaurine (N-(3α,7α,12α-Trihydroxy-24-oxocholan-24-yl)-N-[¹⁸F]fluoromethyl-taurine) and is similar to cholylsarcosine with respect to chemical properties. ¹⁸F has a half time of 2 hours.

In a particular preferred embodiment the compound of Formula 1 comprises the substructure of Formula 3, wherein
R₁, R₃ and R₄ are OH
R₂ is H
R₉ is —CH₂CH₂SO₂OH
R₁₀ is —¹¹CH₃

In a preferred embodiment R₁, R₃ and R₄ are OH in α-position. This compound is equivalent to [N-methyl-¹¹C]cholyltaurine (N-(3α,7α,12α-Trihydroxy-24-oxocholan-24-yl)-N-[¹¹C]methyl-taurine) and is similar to cholylsarcosine with respect to chemical properties.

In a particular preferred embodiment the compound of Formula 1 comprises the substructure of Formula 3, wherein
R₁, R₃ and R₄ are OH
R₂ is H
R₉ is —CH₂COOH
R₁₀ is —CH₂¹⁸F

In a preferred embodiment R₁, R₃ and R₄ are OH in α-position. This compound is equivalent to ¹⁸F-Cholylsarcosine also termed N-(3α,7α,12α-Trihydroxy-24-oxocholan-24-yl)-N-[¹⁸F]fluoromethyl-glycine.

In a most preferred embodiment the compound of Formula 1 comprises the substructure of Formula 3, wherein
R₁, R₃ and R₄ are OH
R₂ is H
R₉ is CH₂COOH
R₁₀ is —¹¹CH₃

In a preferred embodiment R₁, R₃ and R₄ are OH in α-position. This compound is equivalent to [N-methyl-¹¹C]Cholylsarcosine also termed N-(3α,7α,12α-Trihydroxy-24-oxocholan-24-yl)-N-[¹¹C]methyl-glycine.

For the compounds of Formula 1, 2 and 3 it is in one embodiment preferred that H at position 5 is in β-position and H at position 14 is in α-position.

In a preferred embodiment the compound according to the invention is a bile acid derivative. In another preferred embodiment the compound is a bile acid.

A second aspect of the present invention relates to compounds or salts according to Formula 1 for use in an imaging method.

The imaging method may for example be planar scintigraphy, single-photon emission computed tomography (SPECT) or positron emission tomography (PET). In an embodiment the imaging method is SPECT coupled to computed tomography (CT) or magnetic resonance imaging (MRI).). In another embodiment the imaging method is PET coupled to computed tomography (CT) or magnetic resonance imaging (MRI).

A third aspect of the present invention relates to an imaging method comprising:
providing a compound according to Formula 1
administering said compound to an individual
making a radiographic image of a region of interest from said individual.

The radiographic image may in one embodiment be obtained by planar scintigraphy, SPECT or PET. In one embodiment the radiographic image is obtained by SPECT or PET coupled to CT or MRI.

A forth aspect of the present invention relates to a method for diagnosing a disease in an individual said method comprising:
providing a compound according to Formula 1
administering said compound to said individual
making a radiographic image of at least a part of the body from said individual.

A fifth aspect of the present invention relates to a method for determining the biliary excretory function in an individual said method comprising:
providing a compound according to Formula 1
administering said compound to said individual
making a radiographic image of at least a part of the body from said individual.

A sixth aspect of the present invention relates to a method for evaluating the course of disease in an individual said method comprising:
providing a compound according to Formula 1
administering said compound to said individual
making a radiographic image of at least a part of the body from said individual.

A sixth aspect of the present invention relates to a method for evaluating the effect of treatment of a disease in an individual, said method comprising:
providing a compound according to Formula 1
administering said compound to said individual
making a radiographic image of at least a part of the body from said individual.

In one embodiment the method for evaluating the effect of treatment of a disease in an individual further comprise making a first radiographic image at a time point x and comparing said first radiographic image with a second radiographic image obtained from said individual at another time point y. In one embodiment the time point x is before initiating the treatment of said individual and the time point y is after initiating the treatment of said individual. In another embodiment said first radiographic image is taken at a first time point x during treatment of said individual and compared with a second radiographic image obtained form said individual and wherein said second radiographic image is taken at a second time y point during treatment of said individual.

In one embodiment the radiographic image is obtained by PET. The radiographic image may also be obtained by PET coupled to CT or MRI.

In one embodiment the disease referred to in the methods described herein is a hepatic, biliary and/or a gastro-intestinal disorder. The gastro-intestinal disorder may for example be a disorder in the small intestine. The hepatic disorder is in one embodiment a hepatic cancer or a cholestatic disorder.

The term “at least a part of the body” as used herein refers to one or more regions or one or more parts of the body which are scanned. In a preferred embodiment said at least a part of the body is the gastro-intestinal region. In another preferred embodiment said at least a part of the body is at least a part of the hepato-biliary system, such as for example the liver.

The radiolabeled compound may be administered in a dosage of 50-500 megabecquerel (MBq) dependent on the body weight of the individual. It is preferred that compound is administered in a dosage is 3-6 MBq per kilo body weight.

The radiolabeled compound is typically administered by infusion or injections such as for example intravenous administration.

### DESCRIPTION OF DRAWINGS

FIG. 1. Coronal PET/CT images of the time-course of the distribution of ¹¹C—CSar (A) 1 min, (B) 2 min, (C) 15 min, and (D) 38 min after intravenous bolus administration of the tracer (Pig 2). The color-scale is the same for all images.

FIG. 2. Time-activity curves in arterial blood (samples) and in liver tissue, intrahepatic bile ducts, and the choledochus (dynamic PET/CT) after intravenous bolus administration of ¹¹C—CSar (Pig 1).

FIG. 3. Time-activity curves in arterial blood (samples) and in liver tissue, intrahepatic bile ducts, and the choledochus (dynamic PET/CT) after pretreatment with cholyltaurine and intravenous bolus administration of ¹¹C—CSar (Pig 1).

FIG. 4. Coronal images from a dynamic ¹¹C—CSar PET/CT of a patient with cholestasis due to the inherited cholestatic disease BRIC-1. ¹¹C—CSar was given as intravenous infusion. Left image: 5 min after start of infusion, there is still ¹¹C—CSar in the liver In healthy persons ¹¹C—CSar passes through the liver within a few minutes. Right image: 25 min after start of infusion, ¹¹C—CSar has accumulated in the liver and intrahepatic bile ducts. In healthy persons, all ¹¹C—CSar is normally detected in intestines and gallbladder after 25 min.

### DEFINITIONS

The expression ‘C₁₋₆-alk (en/yn)yl’ means a C₁₋₆-alkyl, a C₂₋₆-alkenyl or a C₂₋₆-alkynyl group; wherein:
‘C₁₋₆-alkyl’ refers to a branched or unbranched alkyl groups having from one to six carbon atoms inclusive, including but not limited to methyl, ethyl, 1-propyl, 2-propyl, 1-butyl, 2-butyl, 2-methyl-2-propyl and 2-methyl-1-propyl;
‘C₂₋₆-alkenyl’ designates such groups having from two to six carbon atoms, including one double bond, including but not limited to ethenyl, propenyl, and butenyl; and
‘C₂₋₆-alkynyl’ designates such groups having from two to six carbon atoms, including one triple bond, including but not limited to ethynyl, propynyl and butynyl.

‘C₁₋₈-alk (en/yn)yl’ has the meaning indicated above, but refers to branched or unbranched alkyl group having from one to eight carbon atoms, C₂₋₈-alkenyl having from two to eight carbon atoms including one double bond and C₂₋₈-alkynyl having from two to eight carbon atoms, including one triple bond.

The expression ‘C₃₋₈-cycloalk(en)yl’ means a C₃₋₈-cycloalkyl or a C₃₋₈-cycloalkenyl group; wherein:
‘C₃₋₈-cycloalkyl’ designates a monocyclic or bicyclic carbocycle having three to eight C-atoms, including but not limited to cyclopropyl, cyclopentyl, cyclohexyl etc.; and
‘C₃₋₈-cycloalkenyl’ designates a monocyclic or bicyclic carbocycle having three to eight C-atoms and one double bond, including but not limited to cyclopropenyl, cyclopentenyl, cyclohexenyl, etc.

The terms ‘halogen’ and ‘halo’ means fluoro, chloro, bromo or iodo.

The term ‘hydroxy’ means a OH-group.

The term ‘cyano’ means a CN-group.

In the expression ‘halo-C₁₋₆-alk(en/yn)yl’, ‘halo-C₃₋₈-cycloalk(en)yl’ and ‘hydroxy-C₁₋₆-alk(en/yn)yl’, the terms ‘C₁₋₆-alk(en/yn)yl’, ‘C₃₋₈-cycloalk(en)yl’, ‘hydroxy’ and ‘halo’ are as defined above.

The term ‘aryl’ refers to a carbocyclic aromatic group, such as phenyl or naphthyl, in particular phenyl, and includes both substituted and unsubstituted carbocyclic aromatic groups. Thus, the aryl is optionally substituted with one or more substituents selected from the substituent list as defined herein. Accordingly, the term aryl as used herein means an optionally substituted carbocyclic aromatic group, e.g. phenyl or naphthyl, such that said aromatic group is substituted with one or more substituents selected from the substituent list defined below, e.g., C₁₋₆-alk(en/yn)yl or halogen. The aryl is preferably mono- or bicyclic.

The terms ‘treating’ and ‘treatment’, as used herein, refers to reversing, alleviating, inhibiting the progress of, or preventing the disorder or condition to which such term applies, or one or more symptoms of such disorder or condition.

The term “α-position” as used herein means that the atom or chemical group points down relative to the plan of the steroidal ring system, which is indicated as a dashed line in the chemical drawings.

The term “β-position” as used herein means that the atom or chemical group points up, relative to the plan of the steroidal ring system, which is indicated as a bold line in the chemical drawings.

A wavy or straight line in the chemical drawings indicates that the chemical group can be in either an α-position or a β-position.

### DETAILED DESCRIPTION OF THE INVENTION

The objective of the present invention is to provide compounds that are useful to study the hepatic and intestinal handling of bile acids.

Bile acids (e.g. cholic acid) are amphiphatic steroids formed from cholesterol and conjugated with glycine or taurine in hepatocytes before secretion into the bile canaliculi. They are the major organic component of bile, which flows through the bile ducts into the intestines or is stored in the gallbladder. At the distal small intestines (i.e. the ileum), bile acids are re-absorbed and returned to the liver via portal circulation (i.e. the enterohepatic circulation), where they are taken up by the hepatocytes. The key function of bile acids conjugates is to facilitate intestinal uptake of lipophilic nutrients (e.g. fats and lipid vitamins) as well as to eliminate lipophilic waste products (e.g. bilirubin, excess cholesterol, drug-metabolites, and heavy metals).

Bile acids constitute a large family of molecules, composed of a steroid structure with four rings, a five or eight carbon side-chain terminating in a carboxylic acid, and the presence and orientation of different numbers of hydroxyl groups. The four rings are labeled from left to right (as commonly drawn) A, B, C, and D, with the D-ring being smaller by one carbon than the other three. The hydroxyl groups have a choice of being in 2 positions, either up (or out) termed β (often drawn by convention as a solid line), or down, termed α (seen as a dashed line in drawings). All bile acids have a hydroxyl group on position 3, which was derived from the parent molecule, cholesterol. In cholesterol, the 4 steroid rings are flat and the position of the 3-hydroxyl is β.

Accordingly, an aspect of the present invention relates to a radiolabeled compound comprising the structure of Formula 1:

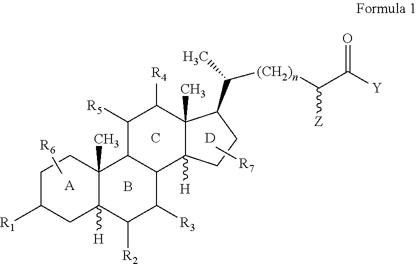

or a salt and/or hydrate thereof;
wherein:
   said compound comprises a steroid structure (ABCD) and at least one radioactive isotope selected from the group consisting of ¹¹C and ¹⁸F
   n is 0, 1, 2 or 3
   X is C or ¹¹C
   Z is H or —CH₃
   Y is selected from the group consisting of OH, OR₈, C₁₋₆-alk(en/yn)yl, NR₉R₁₀
   R₁, R₂, R₃, R₄, R₅, R₆ and R₇ are individually selected from the group consisting of H, OH, C₁₋₆-alk(en/yn)yl, aryl, halo-C₁₋₆-alk(en/yn)yl, C₃₋₈-cycloalk(en)yl, halo-C₃₋₈-cycloalk(en)yl, and hydroxy-C₁₋₆-alk(en/yn)yl, cyano, halogen, oxo, OSO₂OH, —CF₃, and NR₁₁R₁₂
   R₈ is selected from the group consisting of C₁₋₆-alk(en/yn)yl, aryl and C₃₋₈-cycloalk(en)yl, halo-C₁₋₆-alk(en/yn)yl, halo-C₃₋₈-cycloalk(en)yl
   R₉ is selected from the group consisting of H, C₁₋₆-alk(en/yn)yl, —COOH, —CHO, —CH₂COOH, —CH₂COOC₁₋₄alkyl, —CH₂SO₂OH, —CH₂CH₂COOH, —CH₂CH₂COOC₁₋₄ alkyl, —CH₂CH₂SO₂OH, —(CH₂)₁₋₄N⁺R₁₃R₁₄R₁₅, —CH(CH₃)COOH, —CH((CH2)3NHC(NH)NH2)COOH, —CH(CH₂CONH₂)COOH, CH(CH₂COOH)COOH, —CH(CH₂SH)COOH, —CH(CH₂CH₂CONH₂)COOH, —CH(CH₂CH₂COOH)COOH, —CH(CH₂C₃N₂H₃)COOH, —CH(CH(CH₃)CH₂CH₃)COOH, —CH((CH₂)₄NH₂)COOH, —CH((CH₂)₂SCH₃)COOH, —CH(CH₂C₆H₅)COOH, —CH(CH₂OH)COOH, —CH(CH(CH₃)OH)COOH, —CH(CH₂C₈H₆)COOH, —CH(CH₂C₆H₄OH)COOH, —CH(CH(CH₃)₂)COOH
   R₁₀ is selected from the group consisting of H, C₁₋₈-alk(en/yn)yl, ¹¹CH₃, —(CH₂)₁₋₈F, —(CH₂)₁₋₈¹⁸F, CH₂—C₃₋₈-cycloalk(en)yl and CH₂-halo-C₃₋₈-cycloalk(en)yl
   R₁₁ and R₁₂ are individually selected from the group consisting of H, C₁₋₆-alk(en/yn)yl, aryl, C₃₋₈-cycloalk(en)yl
   R₁₃, R₁₄ and R₁₅ are individually selected from the group consisting of C₁₋₈-alk(en/yn)yl

It is preferred that the compound comprising Formula 1 is a bile acid or a bile acid derivative or salts thereof. In a preferred embodiment the compound comprising Formula 1 is a bile acid or a salt thereof.

In one embodiment the steroid structure (ABCD) of the compound comprises one or more double bonds. In one embodiment ring A comprises one double bond which may for example be positioned between position 2 and 3, between position 3 and 4, or between position 4 and 5 of the structure as shown in Formula 1. In one embodiment ring B comprises one double bond which may for example be positioned between position 5 and 6 or between position 6 and 7 of the structure as shown in Formula 1. In one embodiment ring C comprises one double bond, which may for example be positioned between position 10 and 11 of the structure as shown in Formula 1. In another embodiment ring A comprises two double bonds which may for example be positioned between position 2 and 3 and between position 4 and 6 of the structure as shown in Formula 1.

The term (CH₂)ₙ, wherein n=3 means CH₂CH₂CH₂. In one embodiment n=3 or n=2. In another embodiment n=0. In a preferred embodiment n=1.

H at position 5 and H at position 14 can be in either 3-position or α-position. This is indicated by wavy lines in the chemical drawings. In one embodiment H at position 5 is in α-position. In another embodiment H at position 5 is in β-position. In one embodiment H at position 14 is in α-position. In another embodiment H at position 14 is in β-position. In a preferred embodiment H at position 5 is in β-position and H at position 14 is in α-position.

In an embodiment R₁ is selected from the group consisting of H, OH, —C₁₋₆-alk(en/yn)yl, aryl, halo-C₁₋₆-alk(en/yn)yl, —C₃₋₈-cycloalk(en)yl, halo-C₃₋₈-cycloalk(en)yl, hydroxy-C₁₋₆-alk(en/yn)yl, cyano, halogen, OSO₂OH, CF₃, and NR₁₁R₁₂. In an embodiment, R₁ is C₃₋₈-cycloalk(en)yl, such as cyclopropyl. In an embodiment R₁ is C₁₋₆-alk(en/yn)yl, such as C₁₋₆-alkyl, e.g. selected from the group consisting of methyl, ethyl, 1-propyl, 2-propyl. In an embodiment R₁ is halogen, preferably R₁ is fluoro. In another embodiment R₁ is NR₁₁R₁₂ such as NH₂, NHCH₃, N(CH₃)₂. In a preferred embodiment R₁ is selected from the group consisting of H and OH. In a particular preferred embodiment R₁ is OH. R₁ can be in an α-position or a β-position. In a preferred embodiment R₁ is in an α-position. In a most preferred embodiment R₁ is OH in an α-position.

In an embodiment R₂ is selected from the group consisting of H, OH, —C₁₋₆-alk(en/yn)yl, aryl, halo-C₁₋₆-alk(en/yn)yl, —C₃₋₈-cycloalk(en)yl, halo-C₃₋₈-cycloalk(en)yl, hydroxy-C₁₋₆-alk(en/yn)yl, cyano, halogen, OSO₂OH, CF₃, and NR₁₁R₁₂. In an embodiment, R₂ is —C₁₋₆-alk(en/yn)yl, such as —C₁₋₆-alkyl, e.g. selected from the group consisting of methyl, ethyl, 1-propyl and 2-propyl. In a particular embodiment, R₂ is selected from the group consisting of methyl and ethyl. In an embodiment, R₂ is ethyl. Preferably, R₂ is methyl. In an embodiment R₂ is halogen, preferably R₂ is fluoro. In a particular preferred embodiment R₂ is selected from the group consisting of OH or H. In an embodiment R₂ is OH. More preferably R₂ is H. R₂ can be either in α-position or in β-position.

In an embodiment R₃ is selected from the group consisting of H, OH, C₁₋₆-alk(en/yn)yl, aryl, halo-C₁₋₆-alk(en/yn)yl, C₃₋₈-cycloalk(en)yl, halo-C₃₋₈-cycloalk(en)yl, hydroxy-C₁₋₆-alk(en/yn)yl, cyano, halogen, OSO₂OH, CF₃, and NR₁₁R₁₂. In an embodiment, R₃ is C₃₋₈-cycloalk(en)yl, such as cyclopropyl. In an embodiment R₃ is C₁₋₆-alk(en/yn)yl, such as C₁₋₆-alkyl, e.g. selected from the group consisting of methyl, ethyl, 1-propyl, 2-propyl. In an embodiment R₃ is halogen, preferably R₃ is fluoro. In a preferred embodiment R₃ is selected from the group consisting of H and OH. In a preferred embodiment R₃ is H. In a more preferred embodiment R₃ is OH. R₁ can be in an α-position or a β-position. In a particular preferred embodiment R₃ is OH in an α-position.

In an embodiment R₄ is selected from the group consisting of H, OH, C₁₋₆-alk(en/yn)yl, aryl, halo-C₁₋₆-alk(en/yn)yl, C₃₋₈-cycloalk(en)yl, halo-C₃₋₈-cycloalk(en)yl, hydroxy-C₁₋₆-alk(en/yn)yl, cyano, halogen, OSO₂OH, CF₃, and NR₁₁R₁₂. In an embodiment, R₄ is C₃₋₈-cycloalk(en)yl, such as cyclopropyl. In an embodiment R₃ is C₁₋₆-alk(en/yn)yl, such as C₁₋₆-alkyl, e.g. selected from the group consisting of methyl, ethyl, 1-propyl, 2-propyl. In an embodiment R₄ is halogen, preferably R₄ is fluoro. In a preferred embodiment R₄ is selected from the group consisting of H and OH. R₁ can be in an α-position or a β-position. In a particular preferred embodiment R₄ is OH in an α-position.

In an embodiment R₅ is selected from the group consisting of H, OH, C₁₋₆-alk(en/yn)yl, aryl, halo-C₁₋₆-alk(en/yn)yl, C₃₋₈-cycloalk(en)yl, halo-C₃₋₈-cycloalk(en)yl, hydroxy-C₁₋₆-alk(en/yn)yl, cyano, halogen, OSO₂OH, CF₃, and NR₁₁R₁₂. In an embodiment, R₅ is C₁₋₆alk(en/yn)yl, such as C₁₋₆-alkyl, e.g. selected from the group consisting of methyl, ethyl, 1-propyl and 2-propyl. In a particular embodiment, R₅ is selected from the group consisting of methyl and ethyl. In an embodiment, R₅ is ethyl. Preferably, R₅ is methyl. In an embodiment, R₅ is aryl, wherein aryl may be substituted or un-substituted. In a further embodiment, R₅ is aryl. In a further embodiment, R₅ is aryl which is un-substituted. In a further embodiment, R₅ is aryl which is substituted, such as aryl which is substituted with 1, 2 or 3 substituents. In an embodiment, R₅ is aryl which is substituted, such as which 1 or 2 substituents, e.g. with 1 substituent. When R₅ is aryl which is substituted, then the one or more substituents are independently selected from the group consisting of C₁₋₆-alk(en/yn)yl, C₃₋₈-cycloalk(en)yl, OH, SH, NH₂, halogen, cyano, halo-C₁₋₆-alk(en/yn)yl and halo-C₃₋₈-cycloalk(en)yl. In an embodiment, said one or more substituents are independently selected from the group consisting of methyl, OH, SH, NH₂, cyano, and halogen. In a further embodiment, R₅ is phenyl or naphthyl, such as phenyl which is substituted or un-substituted, such as phenyl which is substituted with 1, 2 or 3 substituents wherein said substituents may e.g. be independently selected from the group consisting of C₁₋₆-alk(en/yn)yl, C₃₋₈-cycloalk(en)yl, OH, SH, NH₂, halogen, cyano, halo-C₁₋₆-alk(en/yn)yl and halo-C₃₋₈-cycloalk(en)yl.

In another embodiment, R₅ is C₃₋₈-cycloalk(en)yl, such as cyclopropyl. In an embodiment R₅ is halogen, preferably R₅ is fluoro. In a preferred embodiment R₅ is OH or H. More preferably R₅ is H.

In an embodiment R₆ is selected from the group consisting of H, OH, C₁₋₆-alk(en/yn)yl, aryl, halo-C₁₋₆-alk(en/yn)yl, C₃₋₈-cycloalk(en)yl, halo-C₃₋₈-cycloalk(en)yl, hydroxy-C₁₋₆-alk(en/yn)yl, cyano, halogen, OSO₂OH, CF₃, and NR₁₁R₁₂. In an embodiment, R₆ is C₁₋₆-alk(en/yn)yl, such as C₁₋₆-alkyl, e.g. selected from the group consisting of methyl, ethyl, 1-propyl and 2-propyl. In a particular embodiment, R₆ is selected from the group consisting of methyl and ethyl. In an embodiment, R₆ is ethyl. Preferably, R₅ is methyl. In an embodiment, R₆ is aryl, wherein aryl may be substituted or un-substituted. In a further embodiment, R₆ is aryl. In a further embodiment, R₆ is aryl which is un-substituted. In a further embodiment, R₆ is aryl which is substituted, such as aryl which is substituted with 1, 2 or 3 substituents. In an embodiment, R₆ is aryl which is substituted, such as which 1 or 2 substituents, e.g. with 1 substituent. When R₆ is aryl which is substituted, then the one or more substituents are independently selected from the group consisting of C₁₋₆-alk(en/yn)yl, C₃₋₈-cycloalk(en)yl, OH, SH, NH₂, halogen, cyano, halo-C₁₋₆-alk(en/yn)yl and halo-C₃₋₈-cycloalk(en)yl. In an embodiment, said one or more substituents are independently selected from the group consisting of methyl, OH, SH, NH₂, cyano, and halogen. In a further embodiment, R₆ is phenyl or naphthyl, such as phenyl which is substituted or un-substituted, such as phenyl which is substituted with 1, 2 or 3 substituents wherein said substituents may e.g. be independently selected from the group consisting of C₁₋₆-alk(en/yn)yl, C₃₋₈-cycloalk(en)yl, OH, SH, NH₂, halogen, cyano, halo-C₁₋₆-alk(en/yn)yl and halo-C₃₋₈-cycloalk(en)yl.

In another embodiment, R₆ is C₃₋₈-cycloalk(en)yl, such as cyclopropyl. In an embodiment R₆ is halogen, preferably R₆ is fluoro. In a preferred embodiment R₆ is OH or H. More preferably R₆ is H.

R₆ can be attached to position 1, 2 or 4 in ring A (see Formula 1 with numbers below).

In an embodiment R₇ is selected from the group consisting of H, OH, C₁₋₆-alk(en/yn)yl, aryl, halo-C₁₋₆-alk(en/yn)yl, C₃₋₈-cycloalk(en)yl, halo-C₃₋₈-cycloalk(en)yl, hydroxy-C₁₋₆-alk(en/yn)yl, cyano, halogen, OSO₂OH, CF₃, and NR₁₁R₁₂. In an embodiment, R₇ is C₁₋₆-alk(en/yn)yl, such as C₁₋₆-alkyl, e.g. selected from the group consisting of methyl, ethyl, 1-propyl and 2-propyl. In a particular embodiment, R₇ is selected from the group consisting of methyl and ethyl. In an embodiment, R₇ is ethyl. Preferably, R₇ is methyl. In an embodiment, R₇ is aryl, wherein aryl may be substituted or un-substituted. In a further embodiment, R₇ is aryl. In a further embodiment, R₇ is aryl which is un-substituted. In a further embodiment, R₇ is aryl which is substituted, such as aryl which is substituted with 1, 2 or 3 substituents. In an embodiment, R₇ is aryl which is substituted, such as which 1 or 2 substituents, e.g. with 1 substituent. When R₇ is aryl which is substituted, then the one or more substituents are independently selected from the group consisting of C₁₋₆-alk(en/yn)yl, C₃₋₈-cycloalk(en)yl, OH, SH, NH₂, halogen, cyano, halo-C₁₋₆-alk(en/yn)yl and halo-C₃₋₈-cycloalk(en)yl. In an embodiment, said one or more substituents are independently selected from the group consisting of methyl, OH, SH, NH₂, cyano, and halogen. In a further embodiment, R₇ is phenyl or naphthyl, such as phenyl which is substituted or un-substituted, such as phenyl which is substituted with 1, 2 or 3 substituents wherein said substituents may e.g. be independently selected from the group consisting of C₁₋₆-alk(en/yn)yl, C₃₋₈-cycloalk(en)yl, OH, SH, NH₂, halogen, cyano, halo-C₁₋₆-alk(en/yn)yl and halo-C₃₋₈-cycloalk(en)yl.

In another embodiment, R₇ is C₃₋₈-cycloalk(en)yl, such as cyclopropyl. In an embodiment R₇ is halogen, preferably R₇ is fluoro. In a preferred embodiment R₇ is OH or H. More preferably R₇ is H.

R₇ can be attached to position 15 or 16 in ring D (see Formula 1 with numbers below).

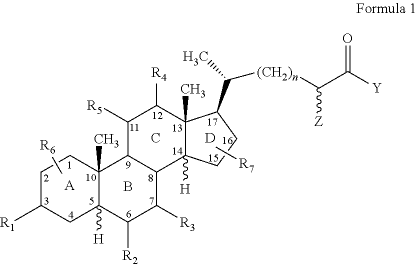

In a specific embodiment R₁, R₃ and R₄ is H or OH. In another specific embodiment R₁ is OH, R₃ is H and R₄ is H. In a preferred embodiment R₁ is OH, R₃ is OH and R₄ is H. In another preferred embodiment R₁ is OH, R₃ is H and R₄ is OH. In a particular preferred embodiment R₁, R₃ and R₄ is OH. In a preferred embodiment R₁, R₃ and R₄ are in α-position. In a particular preferred embodiment R₁, R₃ and R₄ is OH in α-position.

In one embodiment at least one, at least two or more preferable at least three of R₂, R₅, R₆ and R₇ is H. In a specific embodiment R₂, R₅, R₆ and R₇ are H.

Y is selected from the group consisting of OH, OR₈, C₁₋₆-alk(en/yn)yl, NR₉R₁₀. In an embodiment Y is OR₈, where R₈ is selected from the group consisting of C₁₋₆-alk(en/yn)yl, aryl and C₃₋₈-cycloalk(en)yl, halo-C₁₋₆-alk(en/yn)yl, halo-C₃₋₈-cycloalk(en)yl. In an embodiment, R₈ is C₁₋₆-alk(en/yn)yl, such as C₁₋₆-alkyl, e.g. selected from the group consisting of methyl, ethyl, 1-propyl and 2-propyl. In a particular embodiment, R₈ is selected from the group consisting of methyl and ethyl. In an embodiment, R₈ is ethyl. Preferably, R₈ is methyl. In another embodiment, R₇ is C₃₋₈-cycloalk(en)yl, such as cyclopropyl. In an embodiment, R₇ is C₁₋₆-alk(en/yn)yl, such as C₁₋₆-alkyl, e.g. selected from the group consisting of methyl, ethyl, 1-propyl and 2-propyl. In a particular embodiment, R₇ is selected from the group consisting of methyl and ethyl. In an embodiment, R₇ is ethyl. Preferably, R₇ is methyl. In an embodiment, R₇ is aryl, wherein aryl may be substituted or un-substituted. In a further embodiment, R₇ is aryl. In a further embodiment, R₇ is aryl which is un-substituted. In a further embodiment, R₇ is aryl which is substituted, such as aryl which is substituted with 1, 2 or 3 substituents. Substituents are defined elsewhere herein. In an said one or more substituents are independently selected from the group consisting of methyl, OH, SH, NH₂, cyano, and halogen. In a further embodiment, R₇ is phenyl or naphthyl, such as phenyl which is substituted or un-substituted, such as phenyl which is substituted with 1, 2 or 3 substituents.

In another embodiment Y is C₁₋₆-alk(en/yn)yl, such as C₁₋₆-alkyl, e.g. selected from the group consisting of methyl, ethyl, 1-propyl and 2-propyl. In a particular embodiment, Y is selected from the group consisting of methyl and ethyl. In an embodiment, R₇ is ethyl. In another embodiment Y is methyl.

In a preferred embodiment Y is NR₉R₁₀. R₉ is selected from the group consisting of H, C₁₋₆-alk(en/yn)yl, —COOH, —CHO, —CH₂COOH, —CH₂COOC₁₋₄alkyl, —CH₂SO₂OH, —CH₂CH₂COOH, —CH₂CH₂COOC₁₋₄alkyl, —CH₂CH₂SO₂OH, —(CH₂)₁₋₄N⁺R₁₃R₁₄R₁₅, —CH(CH₃)COOH, —CH((CH₂)₃NHC(NH)NH₂)COOH, CH(CH₂CONH₂)COOH, —CH(CH₂COOH)COOH, —CH(CH₂SH)COOH, —CH(CH₂CH₂CONH₂)COOH, —CH(CH₂CH₂COOH)COOH, —CH(CH₂C₃N₂H₃)COOH, —CH(CH(CH₃)CH₂CH₃)COOH, —CH((CH₂)₄NH₂)COOH, —CH((CH₂)₂SCH₃)COOH, —CH(CH₂C₆H₅)COOH, —CH(CH₂OH)COOH, —CH(CH(CH₃)OH)COOH, —CH(CH₂C₈H₆)COOH, —CH(CH₂C₆H₄OH)COOH, —CH(CH(CH₃)₂)COOH. In an embodiment, R₉ is C₁₋₈-alk(en/yn)yl, such as C₁₋₈-alkyl, e.g. selected from the group consisting of methyl, ethyl, 1-propyl and 2-propyl. In a particular embodiment, R₉ is selected from the group consisting of methyl and ethyl. In an embodiment, R₉ is ethyl. In another embodiment, R₉ is methyl. In one embodiment R₉ is H.

In one embodiment R₉ is (CH₂)₁₋₄N⁺R₁₃R₁₄R₁₅, wherein R₁₃, R₁₄ and R₁₅ are individually selected from the group consisting of C₁₋₈-alk(en/yn)yl such as C₁₋₈-alkyl, e.g. selected from the group consisting of methyl, ethyl, 1-propyl and 2-propyl. In a particular embodiment, R₁₃, R₁₄ and R₁₅ are individually selected from the group consisting of methyl and ethyl. In one embodiment at least one, such as at least two of R₁₃, R₁₄ and R₁₅ is methyl. In a preferred embodiment, R₁₃, R₁₄ and R₁₅ are methyl.

In an embodiment R₉ is selected from a group of amino acid side chains (see table 1) consisting of CH₂COOH, —CH(CH₃)COOH, —CH((CH₂)₃NHC(NH)NH₂)COOH—(NHC(NH)NH₂, —CH(CH₂CONH₂)COOH, CH(CH₂COOH)COOH, —CH(CH₂SH)COOH, —CH(CH₂CH₂CONH₂)COOH, —CH(CH₂CH₂COOH)COOH, —CH(CH₂C₃N₂H₃)COOH, —CH(CH(CH₃)CH₂CH₃)COOH, —CH((CH₂)₄NH₂)COOH, —CH((CH₂)₂SCH₃)COOH, —CH(CH₂C₆H₅)COOH, —CH(CH₂OH)COOH, —CH(CH(CH₃)OH)COOH, —CH(CH₂C₈H₆)COOH, —CH(CH₂C₆H₄OH)COOH, —CH(CH(CH₃)₂)COOH.

TABLE 1

| Amino acid scaffold | R₉ (in product)*^{,} ** |
|---|---|
| Glycine | —CH₂COOH |
| Alanine | —CH(CH₃)COOH |
| Arginine | —CH((CH₂)₃NHC(NH)NH₂)COOH |
| | (NHC(NH)NH₂ = guanidino group) |
| Asparagine | —CH(CH₂CONH₂)COOH |
| Asparaginic acid | —CH(CH₂COOH)COOH |
| Cysteine | —CH(CH₂SH)COOH |
| Glutamine | —CH(CH₂CH₂CONH₂)COOH |
| Glutamic acid | —CH(CH₂CH₂COOH)COOH |
| Histidine | —CH(CH₂C₃N₂H₃)COOH |
| | (C₃N₂H₃ = 1H-imidazol-4-yl group) |
| Isoleucine | —CH(CH(CH₃)CH₂CH₃)COOH |
| Leucine | —CH(CH₂CH(CH₃)₂)COOH |
| Lysine | —CH((CH₂)₄NH₂)COOH |
| Methionine | —CH((CH₂)₂SCH₃)COOH |
| Phenylalanine | —CH(CH₂C₆H₅)COOH |
| | (C₆H₅ = phenyl group) |
| Serine | —CH(CH₂OH)COOH |
| Threonine | —CH(CH(CH₃)OH)COOH |
| Tryptophane | —CH(CH₂C₈NH₆)COOH |
| | (C₈NH₆ = 1H-indol-3-yl) |
| Tyrosine | —CH(CH₂C₆H₄OH)COOH |
| | (C₆H₄OH = 4-hydroxyphenyl group) |
| Valine | —CH(CH(CH₃)₂)COOH |

In one embodiment R₉ is selected from the group of amino acid derivatives and R₁₀ is selected from the group consisting of ¹¹CH₃ and ¹⁸FCH₂.

R₉ is in an embodiment selected from the group consisting of CH₂COOC₁₋₄alkyl and —CH₂CH₂COOC₁₋₄alkyl, such as CH₂COOCH₃ and CH₂CH₂COOCH₃. In one embodiment R₉ is selected from the group consisting of COOH, CHO, CH₂COOH, CH₂CH₂COOH and CH₂SO₂OH. In a preferred embodiment R₉ is CH₂SO₂OH. In another preferred embodiment R₉ is selected from the group consisting of CH₂COOH and CH₂CH₂COOH. In a particular preferred embodiment R₉ is CH₂COOH.

R₁₀ is selected from the group consisting of H, C₁₋₈-alk(en/yn)yl, ¹¹CH₃, —(CH₂)₁₋₈F, —(CH₂)₁₋₈¹⁸F CH₂—C₃₋₈-cycloalk(en)yl and CH₂-halo-C₃₋₈-cycloalk(en)yl. In an embodiment, R₁₀ is C₁₋₈-alk(en/yn)yl, such as C₁₋₈-alkyl, e.g. selected from the group consisting of methyl, ethyl, 1-propyl and 2-propyl. In a particular embodiment, R₁₀ is selected from the group consisting of methyl and ethyl. In an embodiment, R₁₀ is ethyl, preferably R₁₀ is methyl. In one embodiment R₁₀ is H. In one embodiment R₁₀ is CH₂F. In a preferred embodiment R₁₀ is CH₂¹⁸F. In a particular preferred embodiment R₁₀ is ¹¹CH₃.

In another preferred embodiment Y is OH.

Formula 2

In further embodiments, the invention relates to compounds or salts of Formula 1, such as compounds comprising the substructure of Formula 2:

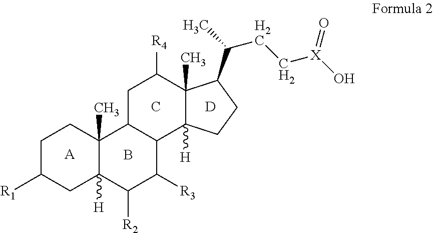

or a salt and/or hydrate thereof;
wherein:
   X is ¹¹C
   R₁, R₂, R₃ and R₄ are individually selected from the group consisting of H, OH, C₁₋₆-alk(en/yn)yl, aryl, halo-C₁₋₆-alk(en/yn)yl, C₃₋₈-cycloalk(en)yl, halo-C₃₋₈-cycloalk(en)yl, and hydroxy-C₁₋₆-alk(en/yn)yl, cyano, halogen, oxo, OSO₂OH, —CF₃, and NR₁₁R₁₂
   R₁₁ and R₁₂ are individually selected from the group consisting of H, C₁₋₆-alk(en/yn)yl, aryl, C₃₋₈-cycloalk(en)yl.

In one embodiment, wherein the compound of Formula 1 comprises the substructure of Formula 2, R₁ is OH. In a preferred embodiment R₁ and R₄ are OH. In a more preferred embodiment R₁, R₃ and R₄ are OH. Preferably R₁, R₃ and R₄ are OH in α-position. In a preferred embodiment R₂ is H.

In a particular preferred embodiment the compound of Formula 1 comprises the substructure of Formula 2, wherein
X is ¹¹C
R₁, R₃ and R₄ are OH
R₂ is H

It is preferred that R₁, R₃ and R₄ are OH in α-position.

Formula 3

In further embodiments, the invention relates to compounds or salts of Formula 1, such as compounds comprising the substructure of Formula 3:

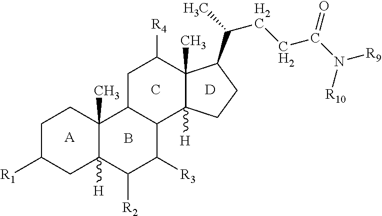

or a salt and/or hydrate thereof;

wherein:
R₁, R₂, R₃ and R₄ are individually selected from the group consisting of H, OH, C₁₋₆-alk(en/yn)yl, aryl, halo-C₁₋₆-alk(en/yn)yl, —C₃₋₈-cycloalk(en)yl, halo-C₃₋₈-cycloalk(en)yl, and hydroxy-C₁₋₆-alk(en/yn)yl, cyano, halogen, oxo, OSO₂OH, —CF₃ and NR₁₁R₁₂
R₉ is selected from the group consisting of H, —C₁₋₆-alk(en/yn)yl, —COOH, —CHO, —CH₂COOH, —CH₂COOC₁₋₄alkyl, —CH₂SO₂OH, —CH₂CH₂COOH, —CH₂CH₂COOC₁₋₄alkyl, —CH₂CH₂SO₂OH, —(CH₂)₁₋₄N⁺R₁₃R₁₄R₁₅
R₁₀ is selected from the group consisting of H, —C₁₋₈-alk(en/yn)yl, —¹¹CH₃, —(CH₂)₁₋₈F, —(CH₂)₁₋₈¹⁸F, —CH₂—C₃₋₈-cycloalk(en)yl, —CH₂— halo-C₃₋₈-cycloalk(en)yl
R₁₁ and R₁₂ are individually selected from the group consisting of H, C₁₋₈-alk(en/yn)yl, aryl, C₃₋₈-cycloalk(en)yl
R₁₃, R₁₄ and R₁₅ are individually selected from the group consisting of —C₁₋₈-alk(en/yn)yl

In one embodiment, wherein the compound of Formula 1 comprises the substructure of Formula 3, R₁, R₂, R₃ and R₄ are individually selected from the group consisting of H and OH. In a preferred embodiment R₁, R₃ and R₄ are OH and R₂ is H. In a more preferred embodiment R₁, R₃ and R₄ are OH in α-position and R₂ is H.

In one embodiment, wherein the compound of Formula 1 comprises the substructure of Formula 3, R₉ is CH₂COOH and R₁₀ is —CH₂¹⁸F. In a particular preferred embodiment, wherein the compound of Formula 1 comprises the substructure of Formula 3, R₉ is CH₂COOH and R₁₀ is ¹¹CH₃.

In one embodiment, wherein the compound of Formula 1 comprises the substructure of Formula 3, R₉ is CH₂CH₂SO₂OH and R₁₀ is —CH₂¹⁸F. In a more embodiment embodiment, wherein the compound of Formula 1 comprises the substructure of Formula 3, R₉ is —CH₂CH₂SO₂OH and R₁₀ is ¹¹CH₃.

In a particular preferred embodiment the compound of Formula 1 comprises the substructure of Formula 2, wherein
R₁, R₃ and R₄ are OH
R₂ is H
R₉ is CH₂CH₂SO₂OH
R₁₀ is —CH₂¹⁸F

In a preferred embodiment R₁, R₃ and R₄ are OH in α-position. This compound is equivalent to ¹⁸F-cholyltaurine (N-(3α,7α,12α-Trihydroxy-24-oxocholan-24-yl)-N—[¹¹C]fluoromethyl-taurine) and is similar to cholylsarcosine with respect to chemical properties. ¹⁸F has a half time of 2 hours.

In a particular preferred embodiment the compound of Formula 1 comprises the substructure of Formula 2, wherein
R₁, R₃ and R₄ are OH
R₂ is H
R₉ is CH₂CH₂SO₂OH
R₁₀ is ¹¹CH₃

In a preferred embodiment R₁, R₃ and R₄ are OH in α-position. This compound is equivalent to [N-methyl-¹¹C]cholyltaurine (N-(3α,7α,12α-Trihydroxy-24-oxocholan-24-yl)-N—[¹¹C]methyl-taurine) and is similar to cholylsarcosine with respect to chemical properties.

In a particular preferred embodiment the compound of Formula 1 comprises the substructure of Formula 2, wherein
R₁, R₃ and R₄ are OH
R₂ is H
R₉ is CH₂COOH
R₁₀ is CH₂¹⁸F

In a preferred embodiment R₁, R₃ and R₄ are OH in α-position. This compound is equivalent to ¹⁸F-Cholylsarcosine also termed N-(3α,7α,12α-Trihydroxy-24-oxocholan-24-yl)-N—[¹⁸F]-glycine.

In a most preferred embodiment the compound of Formula 1 comprises the substructure of Formula 2, wherein
R₁, R₃ and R₄ are OH
R₂ is H
R₉ is CH₂COOH
R₁₀ is ¹¹CH₃

In a preferred embodiment R₁, R₃ and R₄ are OH in α-position. This compound is equivalent to [N-methyl-¹¹C]Cholylsarcosine also termed N-(3α,7α,12α-Trihydroxy-24-oxocholan-24-yl)-N—[¹¹C]methyl-glycine. Cholylsarcosine (CSar; N-methyl-cholylglycine) is an analog of the endogenous bile acid conjugate cholylglycine, which is derived from the bile acid cholic acid and the amino acid glycine. CSar is based in a natural occurring bile acid, is non-toxic to humans and undergoes an enterohepatic circulation without hepatic or intestinal biotransformation in humans. Further, the more —OH-groups present in the compound the more active transport (i.e. movement against its concentration gradient) of the compound through cell membranes including liver cells. Accordingly, compounds having more —OH groups are preferred.

The relatively short half time of ¹¹C (20 minutes) may be an advantage when compared to the half time of for example ¹⁸F (109 minutes) for repeating a scan on an individual, since the radioactivity concentration in the individual will decrease to <3.5% in the course of 5 half times for ¹¹C (100 minutes), whereas a study using ¹⁸F cannot in practice be repeated within the same day.

Stereochemistry of the Compounds of the Invention

Compounds, hydrates, salts or prodrugs of the present invention may contain chiral centers and therefore may exist in different enantiomeric and diastereomeric forms. This invention relates to all optical isomers and all stereoisomers of the compounds or salts of the present invention, both as racemic mixtures and as individual enantiomers and diastereoismers ((+)- and (−)-optically active forms), and mixtures thereof, and to all pharmaceutical compositions and methods of treatment defined herein that contain or employ them, respectively. Individual isomers can be obtained by known methods, such as optical resolution, optically selective reaction, or chromatographic separation in the preparation of the final product or its intermediate.

Methods for Synthesizing the Compounds of the Invention

In one embodiment the radiolabeled compounds comprising formula 1 is synthesized using a three-step procedure as exemplified in Scheme 1. Normally, radiolabeled compounds having a relatively short half time, e.g. 20 minutes for ¹¹C-labeled substances, and used for scanning methods such as for example PET are synthesized using a one- or two-step procedure. Methods comprising more than two steps have been considered taking too much time for the labelling of compounds having short half times. However, it has been found that the three-step procedure as exemplified in Scheme 1 is surprisingly fast and the three-step procedure can therefore be used for labelling compounds with isotopes having a short half time such as for example ¹¹C having a half time of 20 minutes. In one preferred embodiment [N-methyl-¹¹C]Cholylsarcosine and/or ¹⁸F-Cholylsarcosine (i.e. N—[¹⁸F]fluoromethyl-cholylglycine) is synthesised using a method comprising three synthesis steps. Such a method is illustrated in Scheme 1 and 2. It is appreciated that compounds wherein R₉ comprises —COOH is synthesized using a three-step procedure.

Methods

Another aspect of the present invention relates to a compound comprising Formula 1 for use in an imaging method. The compound may be any of the compounds described herein and comprising Formula 1. Accordingly, the radiolabeled compounds according to the invention may be referred to as radiolabeled tracers.

The imaging method is in a preferred embodiment PET. Radiolabeled tracers are administered intravenously (or as an inhalation) and the emitted radiation is captured by external cameras to form 3-D images

PET is nuclear medicine imaging technique that produces a 3-D images or pictures of radioactivity concentrations in the body. To conduct the scan, a relatively short-lived radioactive tracer is injected into a living subject (usually intravenously). The camera comprises rings of crystals, which detect pairs of gamma rays emitted indirectly by the administered radiolabeled tracer; the recorded data are corrected for radioactive half life back to the time of tracer injection and 3-D images of radioactivity concentration within the body are then constructed by computer analysis.

In modern scanners, 3-D PET imaging and CT scan are integrated in one scanner and thus performed during the same session. When a static scan is conducted, there is a waiting period while the radiolabeled tracer distributes and becomes concentrated in tissues of interest; then the subject is placed in the camera and PET recordings performed in a prefixed time interval, typically 20-30 min for scans of the body from the skull to mid-tights. Today, the molecule most commonly used for this purpose is ¹⁸F-FDG, a sugar, for which the waiting period is typically an hour. For a dynamic scan, the PET recording is performed during and immediately following tracer administration, and data are reconstructed to provide time-courses of radioactivity concentrations in the part of the body examined. The time period for such a scan is typically 40-60 min and data can be used to generate images of for example metabolic function or blood perfusion of the organ examined.

PET scans are increasingly read alongside CT or magnetic resonance imaging (MRI) scans, with the combination giving both anatomic and metabolic information. Because PET imaging is most useful in combination with anatomical imaging, such as CT, modern PET scanners are now available with integrated high-end multi-detector-row CT scanners. Because the two scans can be performed in immediate sequence during the same session, with the patient not changing position between the two types of scans, the two sets of images are more-precisely registered, so that areas of abnormality on the PET imaging can be more perfectly correlated with anatomy on the CT images.

Accordingly, in one preferred embodiment PET is combined with computed tomography (CT). In another embodiment PET is combined with magnetic resonance imaging (MRI).

The present invention also relates to imaging methods wherein a compound according to the invention is administered into an individual. A radiographic image is subsequently obtained using nuclear imaging techniques such as for example PET.

Accordingly, a further aspect of the invention relates to an imaging method comprising:
providing a compound as described herein
administering said compound to an individual
making a radiographic image of at least a part of the body from said individual

The imaging method may for example be used for diagnosing a disease in an individual. Accordingly, an aspect of the invention relates to a method for diagnosing a disease in an individual said method comprising:
providing a compound as described herein
administering said compound to said individual
making a radiographic image of at least a part of the body from said individual

A further aspect of the invention relates to method for determining the biliary excretory function in an individual said method comprising:
providing a compound as described herein
administering said compound to said individual
making a radiographic image of at least a part of the body from said individual

The biliary excretory function may for example be determined in healthy individuals to characterize and quantify normal biliary excretory function or to determine whether pharmaceuticals or drugs influence the biliary excretory function. Thus, the method can be used for determining the toxicity of medicaments/drugs. The method can also be used for determining or quantifying the biliary excretory function of individuals suffering from a disease as described herein.

The compound of the present invention may also be used to evaluate the course of disease in an individual or for evaluating the effect of treatment of a disease in an individual.

Accordingly, a further aspect of the invention relates to a method for evaluating the course of disease in an individual said method comprising:
providing a compound as described herein
administering said compound to said individual
making a radiographic image of at least a part of the body from said individual.

Another aspect of the invention relates to method for evaluating the effect of treatment of a disease in an individual, said method comprising:
providing a compound as described herein
administering said compound to said individual
making a radiographic image of at least a part of the body from said individual

The method may for example comprise making a first radiographic image at a time point x and comparing said image with a second radiographic image obtained from said individual at another time point y, wherein x is an earlier time point than y. The time point x may for example be before initiating the treatment of said individual, whereas y is after initiating the treatment of said individual. Alternatively a radiographic image is taken at a first time point x during treatment of said individual and compared with a second radiographic image obtained form said individual and wherein said second radiographic image is taken at a second time y point during treatment of said individual.

The individual as referred to herein may for example be an animal such as a mammal, porcine, bovine, horse, monkey, dog, rodent, mouse, rat, vertebrate or reptile. Preferably the individual is a human.

In the methods of the present invention the radiographic image is made of “at least a part of the body” from the individual. The term “at least a part of the body” as used herein refers to one or more regions or one or more parts of the body which are scanned. The at least a part of the body may for example be the entire body. In a dynamic scan only one part of the body is scanned after injection of the radiolabeled compound. The at least a part of the body which is scanned may for example include the liver, bile ducts and the aorta. In a preferred embodiment the at least a part of the body is the gastro-intestinal region. In another preferred embodiment the at least a part of the body is at least a part of the hepato-biliary system such as for example the liver.

In a preferred embodiment the radiographic image is obtained by PET. In one embodiment the radiographic image is obtained by PET coupled to CT or MR.

In a preferred embodiment the disease as referred to herein is a hepatic, biliary and/or a gastro-intestinal disorder.

The hepatic disorder may for example be hepatic cancer or a cholestatic disorder. Examples of hepatic disorders with intrahepatic cholestasis include acute hepatitis, drug-induced liver disease, primary biliary cirrhosis (PBC), viral hepatitis B or C with or without cirrhosis, alcoholic liver disease, cholestasis of pregnancy, radiation-induced damage, and primary sclerosing cholangitis (PSC). Examples of biliary diseases are gallstone diseases, biliary atresia and damages of the bile ducts caused by surgery. Further examples of cholestatic hepatic diseases are familial intrahepatic cholestasis (PFIC) and severe be salt export pump (BSEP) deficiency, which is a hereditary cholestatic condition. Hepatic cancers include primary cancers such as example hepatocellular carcinoma and cholangiocarcinoma as well as secondary liver cancers such as metastases from colorectal cancers and neuroendocrine tumours.

The gastro-intestinal disorder may for example be a disorder in the small intestine as for example Crohn's disease.

The radiolabeled compound may be administered in a dosage of 50-500 megabecquerel (MBq) dependent on the body weight of the individual. It is preferred that compound is administered in a dosage is 3-6 MBq per kilo body weight.

The radiolabeled compound is typically administered by infusion or injections such as for example intravenous administration.

The present invention may also be used for analysing blood samples, urine samples and/or bile samples for the presence or absence of the radiolabeled radiotracer.

Pharmaceutical Formulations

The compounds, hydrates or salts of the present invention can be presented in the form of a pharmaceutical formulation. Accordingly, the present invention further provides a pharmaceutical formulation, which comprises a compound of the present invention or a pharmaceutically acceptable salt thereof, as herein defined, and a pharmaceutically acceptable carrier thereof. The pharmaceutical formulations may be prepared by conventional techniques, e.g. as described in Remington: The Science and Practice of Pharmacy 2005, Lippincott, Williams & Wilkins.

The compounds of the present invention may be formulated for parenteral administration such as intravenous administration and may be presented in unit dose form in ampoules, pre-filled syringes, small volume infusion or in multi-dose containers. The compositions may take such forms as suspensions, solutions, or emulsions in oily or aqueous vehicles, for example solutions in aqueous polyethylene glycol. Examples of oily or non-aqueous carriers, diluents, solvents or vehicles include propylene glycol, polyethylene glycol, vegetable oils (e.g., olive oil), and injectable organic esters (e.g., ethyl oleate), and may contain agents such as preserving, wetting, emulsifying or suspending, stabilizing and/or dispersing agents.

Preferably, the formulation will comprise about 10-250 MBq per millilitre by weight of the active ingredient(s) with the remainder consisting of suitable pharmaceutical excipients as described herein.

Pharmaceutically acceptable salts of the instant compounds, where they can be prepared, are also intended to be covered by this invention. These salts will be ones which are acceptable in their application to a pharmaceutical use. By that it is meant that the salt will retain the biological activity of the parent compound and the salt will not have untoward or deleterious effects in its application and use in treating diseases.

Pharmaceutically acceptable salts are prepared in a standard manner. If the parent compound is a base it is treated with an excess of an organic or inorganic acid in a suitable solvent. If the parent compound is an acid, it is treated with an inorganic or organic base in a suitable solvent.

The compounds of the invention may be administered in the form of an alkali metal or earth alkali metal salt thereof, concurrently, simultaneously, or together with a pharmaceutically acceptable carrier or diluent, especially and preferably in the form of a pharmaceutical composition thereof, whether by oral, rectal, or parenteral (including subcutaneous) route, in an effective amount.

Examples of pharmaceutically acceptable acid addition salts for use in the present inventive pharmaceutical composition include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic acids, and arylsulphonic, for example.

### EXAMPLES

### Methods for Synthesizing the Compounds of the Invention

Cyclotron Production of Applied Radionuclides

The applied radionuclides are indicated in column 1 and are produced according to the nuclear reactions described in column 2 by cyclotron bombardment using the target gases indicated in column 3. The radionuclides produced are obtained from the cyclotron as the products indicated in column 4.

| Radionuclide | Nuclear reaction | Target gas | Target product |
|---|---|---|---|
| Carbon-11 (¹¹C) | ¹⁴N(p, α)¹¹C | N₂ (+O₂) | ¹¹CO₂ (gas) |
| | | N₂ (+H₂) | ¹¹CH₄ (gas) |
| Fluorine-18 (¹⁸F) | ¹⁸O(p, n)¹⁸F | H₂¹⁸O | ¹⁸F⁻ (aq. solution) |

Preparation of [¹¹C]Methyl Iodide (¹¹CH₃I) and [¹¹C]Methyl Triflate (¹¹CH₃OTf)

¹¹CH₃I is prepared from ¹¹CO₂ or ¹¹CH₄ by a standard liquid phase or gas phase procedure using a commercially available synthesis system (e.g. MeI-Plus® from BioScan, or TRACERlab™ FX C Pro or TRACERlab™ FX MeI from GE Healtcare). In the liquid phase procedure, cyclotron produced ¹¹CO₂ is initially trapped on molecular sieves. The trapped ¹¹CO₂ is then reduced by LiAlH₄ in THF to ¹¹CH₃OH, which is subsequently reacted in the system with aqueous hydrogen iodide to give ¹¹CH₃I. In the gas phase procedure, ¹¹CH₄ (produced in the cyclotron or in the synthesis system by hydrogenation of cyclotron produced ¹¹CO₂ over a nickel catalyst) is concentrated in a cooling trap before reaction with molecular iodide to give ¹¹CH₃I. The formed ¹¹CH₃I is transferred directly to a reaction vial or transformed into ¹¹CH₃OTf. ¹¹CH₃OTf is prepared by passing ¹¹CH₃I over a column of silver triflate with heating.

Preparation of [¹¹C]hydrogencyanide (H¹¹CN)

Carbon-11 labeled hydrogen cyanide (H¹¹CN) is prepared using a standard system (supplied e.g. by GE Healtcare) in connection to the cyclotron. In this system, ¹¹CO₂ is initially hydrogenation over a nickel catalyst to give ¹¹CH₄. The ¹¹CH₄ is subsequently reacted with ammonia over a platinum catalyst at high temperature (1000° C.) to give H¹¹CN. The H¹¹CN is trapped in the reaction vial immediately before the radiosynthesis.

Preparation of [¹⁸F]fluoride (⁸F)

The ¹⁸F is separated from the aqueous solution received from the cyclotron by standard ion-exchange chromatography using a solid-phase extraction quaternary ammonium carbonate cartridge (e.g. Sep-Pak® Light Accell Plus QMA cartridge from Waters). The ¹⁸F is washed of the cartridge using an aqueous or an aqueous acetonitrile solution of potassium carbonate. The isolated potassium salt of ¹⁸F⁻ is dried by azeotropic distillation using dry acetonitrile. The dried potassium salt of ¹⁸F⁻ is complexed in a reaction vial with Kryptofix® 2.2.2 (K222) in dry acetonitrile before being used for synthesis or for transformation into ¹⁸FCH₂Br or ¹⁸FCH₂OTf.

Preparation of ¹⁸FCH₂Br and ¹⁸FCH₂OTf

¹⁸FCH₂Br and ¹⁸FCH₂OTf are prepared as described in the literature (Iwata R et al. Appl Radiat Isot 2002; 57:347-352): The complex ¹⁸F⁻/K222/K⁺ in acetonitrile, prepared as described above, is added CH₂Br₂ in dry acetonitrile and refluxed. The formed ¹⁸FCH₂Br is purified by passing it though successive solid-phase extraction silica cartridges (e.g. Sep-Pak® Plus silica cartridges from Waters) before being used for synthesis or for transformation into ¹⁸FCH₂OTf. ¹⁸FCH₂OTf is formed by passing ¹⁸FCH₂Br over a column of silver triflate with heating.

### Example 1

### Preparation of ¹¹C-Cholylsarcosine (N-(3α,7α,12α-Trihydroxy-24-oxocholan-24-yl)-N—[¹¹C]methyl-glycine)

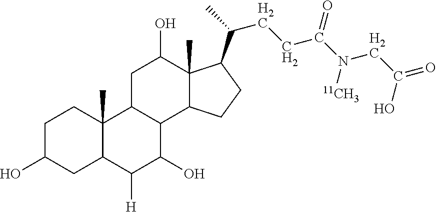 [SMILES: C[C@H](CCC(=O)N([11CH3])CC(=O)O)[C@H]1CCC2C3C(O)CC4CC(O)CC[C@]4(C)C3CC(O)[C@@]21C]

Radiochemistry

N-(3α,7α,12α-Trihydroxy-24-oxocholan-24-yl)-N—[¹¹C]methyl-glycine (¹¹C—CSar) was prepared by the three-step radiosynthesis as illustrated in Scheme 1 below.

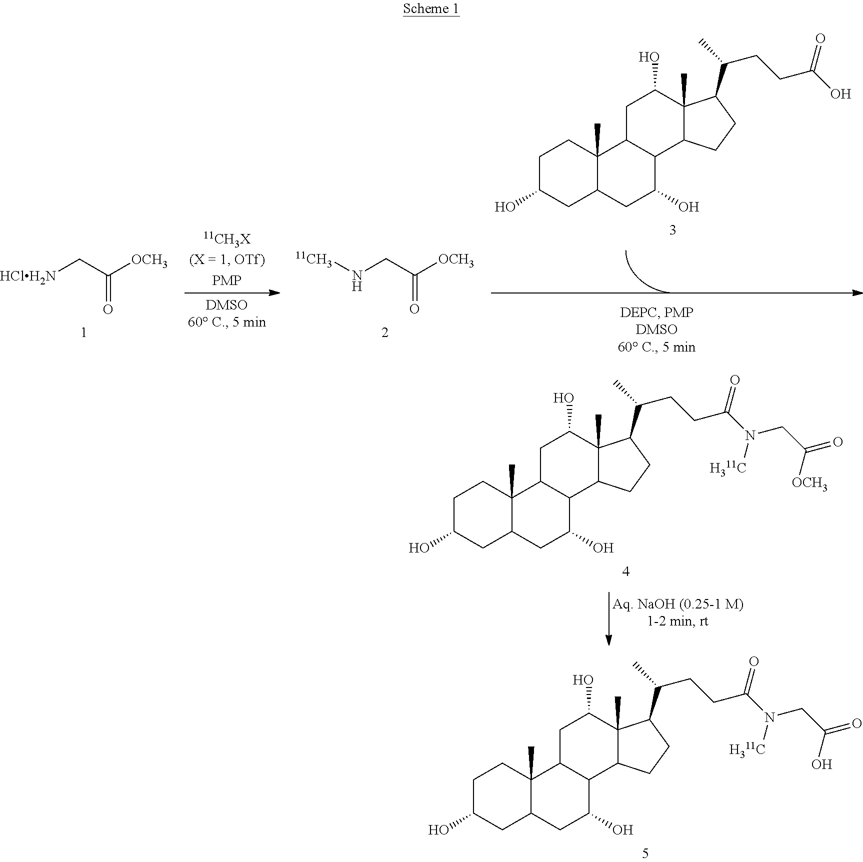

The ¹¹CH₃I is delivered to a mixture of glycine methyl ester hydrochloride 1 (0.8±0.2 mg, 6±2 μmol) and PMP (5 μl, 28 μmol) in anhydrous DMSO (300 μl) at room temperature. The sealed reaction vial is heated at 60° C. for 5 min. Solutions of cholic acid 3 (12 mg, 29 μmol) in anhydrous DMSO (250 μl) and DEPC (5 μl, 29 μmol) in anhydrous DMSO (150 μl) are then successively added. The reaction mixture is heated again at 60° C. for 5 min and then quenched sterile water (2 ml). The crude ¹¹C—CSar methyl ester 4 is purified by reverse phase HPLC on a Waters®-XTerra® Prep RP₁₈ OBD™ (5 μm, 19×100 mm) column with 33-40% acetonitrile in sterile aqueous NaH₂PO₄ (70 mM) as mobile phase (flow 20 ml/min; λ=220 nm). The fraction corresponding to 4 is collected and diluted with a large volume of sterile water (50 ml) before passed slowly over a C18 or C8 solid phase extraction cartridge on which the 4 is trapped. The cartridge is subsequently washed with sterile water (15 ml) before 4 is eluted from the cartridge using sterile ethanol (1 ml). Sterile aqueous 0.25-1 M NaOH (2 ml) is then passed through the cartridge into the ethanolic solution. The alkaline mixture is allowed to stand for 1-2 min at room temperature, then neutralized with sterile aq. NaH₂PO₄ or aq. citrate buffer (7 ml), and passed through a sterile filter (0.22 μm) into a sterile product vial. The radioactivity of the final product 5 is measured using a dose calibrator and a small sample (˜0.3 ml) is withdrawn for quality control analysis. The identity of ¹¹C—CSar 5 is confirmed using LC-radio-MS and compared with reference material (i.e. unlabeled cholylsarcosine prepared as described below).

The final deprotective hydrolysis of the isolated ¹¹C—CSar methyl ester using aqueous NaOH (0.25-1 M) proceeded with full conversion at room temperature for 1-2 min to give ¹¹C—CSar (5).

The three-step radiosynthesis provided 0.56-1.09 GBq of ¹¹C—CSar (5) with a radiochemical yield of 13±3% (mean±SD; n=7, decay-corrected) and with radiochemical purity >99% within 40 min. The radiochemical yield of the intermediate ¹¹C-cholylsarcosine methyl ester 4 was approximately 20% and the deprotection proceeded with full conversion to give ¹¹C—CSar exclusively. In its final formulation, the tracer showed no alterations in chemical or radiochemical purity for up to 2.5 h after the end of the synthesis.

Optimization of Reaction Conditions and Method for the Radiosynthesis of ¹¹C-Cholylsarcosine (¹¹C—CSar, 5)

In the presented three-step radiosynthesis of ¹¹C-cholylsarcosine (¹¹C—CSar, 5), the first two steps (i.e. methylation and coupling) were performed as a one-pot procedure in DMSO at 60° C. with PMP as the auxiliary base and DEPC as the coupling reagent. Under these conditions, the reaction proceeded to give ¹¹C—CSar methyl ester 4 with a radiochemical yield (RCY) of 20%. This yield was determined by analytical HPLC (retention time of ¹¹C—CSar methyl ester: 4.6 min) of the crude reaction mixture using the Luna® column with 40% acetonitrile in aqueous NaH₂PO₄ (70 mM) as eluent (isocratic, 2.5 mL/min) and obtained after optimization of reaction parameters including solvent, concentration, auxiliary base, temperature and reaction time as described below:

Solvent:

A screening of solvents revealed that DMF provided a similar RCY as DMSO (19%), while MeCN failed to provide any ¹¹C—CSar methyl ester 4 presumably due to the low solubilities of the glycine methyl ester hydrochloride and cholic acid in this solvent. Lower yields are generally obtained if the solvent is not dry.

Concentrations:

Changes in the ratios of the reagents were not observed to have any significant effect on the RCY.

Auxiliary Base:

As an auxiliary base, TMP performed similar (20% RCY) to PMP, while Et₃N resulted in a slightly lower RCY (15%). Addition of PMP in two portions (1.5 μL for the methylation and 3.5 μL for the coupling) rather than one (5 μL) did not improve the RCY.

Reaction Temperature:

A lower RCY was observed when the reaction was carried out at room temperature (6%) rather than at 60° C. However, no improvement in RCY was observed when the reaction was performed at 80° C. and at higher temperature significant decomposition of reagents was observed.

Reaction Time:

Reaction times (up to 20 min) for the methylation or coupling did not improve the overall RCY of the two-step one-pot preparation of the ¹¹C—CSar methyl ester.

Equipment:

A significantly higher RCY (approx. 50%) is obtained when performing the radiosynthesis on an automated (and thoroughly pre-dried) synthesis system such as the GE Tracerlab™ FX_{C} Pro system.

Preparation of Unlabeled Cholylsarcosine for Use as Reference Material

A solution of cholic acid (2.1 g; 5.1 mmol) and sarcosine methyl ester hydrochloride (0.7 g; 5.0 mmol) in dry DMF (35 ml) was cooled to 0° C., and DEPC (0.83 ml; 5.5 mmol) was added. Dry Et₃N (3.5 ml; 25 mmol) was then added dropwise over 10 min, and the mixture was stirred under argon for 45 min at 0° C., then overnight at room temperature. Full conversion of cholic acid was observed by TLC (10% methanol in DCM; R_{f}=0.25). The precipitated salts were filtered off and the filtrate was concentrated by reduced pressure to give a yellow oil. The crude product was purified by silica gel chromatography (eluent: 8% EtOAc in hexane) to give cholylsarcosine methyl ester as a white solid (0.97 g; 39%). ¹H NMR (Varian AS 400 running at 400 MHz, CDCl₃, 25° C., 8 mg/ml, chemical shifts (δ) are reported relative to residual signals of CDCl₃ (δ=7.26)). Two sets of resonances (rotamers) were observed in a 4:1 ratio.

When resolved, 6 of the minor resonance are given in square brackets: δ 4.12 (s, 2H) [4.06 (d, J=3.9 Hz, 2H)], 3.97 (m, 1H), 3.84 (m, 1H), [3.78 (s, 3H)] 3.72 (s, 3H), 3.44 (m, 1H), 3.08 (s, 3H) [2.96 (s, 3H)], 2.41 (m, 1H), 2.17-2.23 (m, 5H), 1.25-1.91 (m, 20H), 1.10 (m, 1H), 1.01 (d, J=6.1 Hz, 3H), 0.88 (s, 3H), 0.68 (s, 3H) ppm.

To the cholylsarcosine methyl ester (50 mg; 0.10 mmol) was added methanolic KOH (5% w/w; 2 ml) and the solution was stirred in a closed vial at 70° C. for 30 min. Full conversion of the methyl ester was observed by TLC (25% methanol in DCM; R_{f}=0.05). The solvent was evaporated under a stream of N₂ gas to give a clear oil. The oil was re-dissolved in water (5 mL) and the solution was acidified with 5% aq. H₂SO₄ under stirring. The acidic opaque solution was left at room temperature to allow for a white precipitate to form. After filtration and drying under vacuum, pure unlabeled CSar was obtained as a white solid (27 mg; 56%). ¹H NMR (Varian AS 400 running at 400 MHz, CD₃OD, 25° C., 7 mg/ml, chemical shifts (6) are reported relative to residual signals of CD₃OD (δ=3.31)). Two sets of resonances (rotamers) were observed in a 2.5:1 ratio. When resolved, δ of the minor resonance are given in square brackets: δ [4.18 (s, 2H)] 4.09 (s, 2H), 3.95 (m, 1H), 3.80 (m, 1H), 3.35 (m 1H), 3.12 (s, 3H) [2.94 (s, 3H)], 2.49 (m, 1H), 2.22-2.35 (m, 4H), 1.72-2.01 (m, 8H), 1.30-1.71 (m, 12H), 1.14 (m, 1H), 1.06 (d, J=6.5 Hz, 3H) [1.01 (d, J=6.4 Hz, 3H)], 0.97 (m, 1H), 0.92 (s, 3H), 0.72 (d, J=4.8 Hz, 3H) ppm. ESI-MS (Bruker Daltonics HCT Plus (ion trap), negative ionization mode, capillary voltage +4.5 kV): 478.3 m/z [M-H]⁻.

### Example 2

### Preparation of N-([¹⁸F]fluoromethyl)cholylglycine (N-(3α,7α,12α-Trihydroxy-24-oxocholan-24-yl)-N—[¹⁸F]fluoromethyl-glycine)

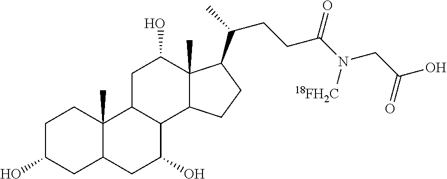

Radiosynthesis

The following radiosynthesis (Scheme 2) cover the compounds according to Formula 1 and Formula 3, wherein R₉ is a COOH group and R₁₀ is CH₂¹⁸F.

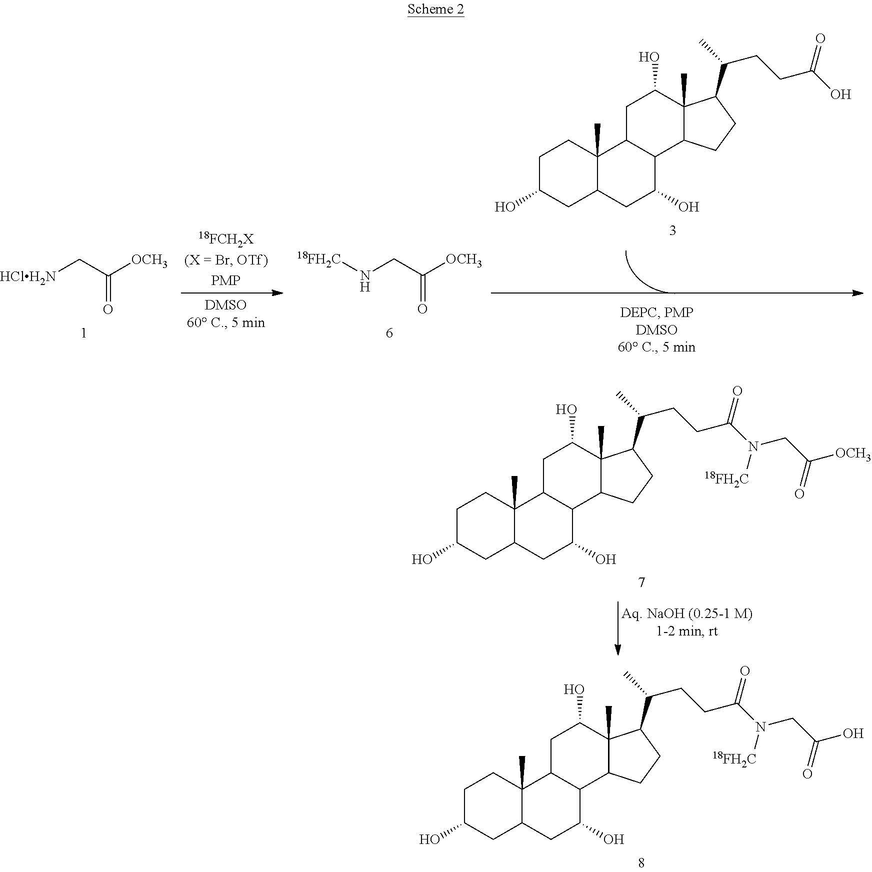

¹⁸FCH₂X (X=Br or OTf) is added to a mixture of glycine methyl ester hydrochloride 1 (0.8 mg; 6 μmol) and PMP (5 μl; 28 μmol) in dry DMSO (300 μl) at room temperature. The sealed reaction vial is heated in an oil bath at 60° C. for 5 min. The vial is removed from the oil bath and solutions of cholic acid 3 (12 mg; 29 μmol) in dry DMSO (250 μl) and DEPC (5 μl; 29 μmol) in dry DMSO (150 μl) are successively added. The reaction mixture is heated at 60° C. for 5 min and then quenched with water or aq. ethanol (4 ml). The formed compound 7 is purified by preparative HPLC using conditions determined by the stilled chemist. The fraction containing 7 is collected and diluted with water (50 ml), before passed slowly over a C₁₈ or C₈ solid phase extraction cartridge (preconditioned with 10 ml ethanol, followed by 10 ml water) on which 7 is trapped. The cartridge is washed with water (15 ml) before 7 is eluted from the cartridge using ethanol (1 ml). Aqueous 0.25-1M NaOH (2 ml) is then passed through the cartridge into the ethanolic solution. The alkaline mixture is allowed to stand for 1-2 min at room temperature, before finally neutralized with aq. NaH₂PO₄ or citrate buffer (7 ml) to give a neutral aq. solution of the final product 8.

### Example 3

### Preparation of N—[¹¹C]Methyltaurine Conjugated Bile Acids—Exemplified by the Preparation of N—([¹¹C]Methyl)Cholyltaurine (N-(3α,7α,12α-Trihydroxy-24-Oxocholan-24-Yl)-N—[¹¹C]Methyl-Taurine)

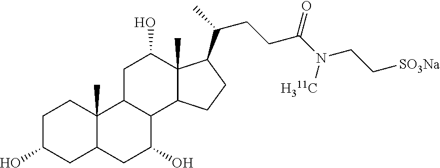

Radiosynthesis

The following radiosynthesis (Scheme 3) describes the preparation of N-([¹¹C]methyl)cholyltaurine (i.e. Formula 3 where R₁, R₃ and R₄ are α-OH, R₂ is H, R₉ is CH₂CH₂SO₃Na, and R₁₀ is ¹¹CH₃), but it has also been shown to be applicable (with minor modifications as described in the text below) for the radiosynthesis of other similar compounds such as:

N-([¹¹C]methyl)chenodeoxycholyltaurine (i.e. Formula 3 where R₁ and R₃ are α-OH, R₂ and R₄ are H, R₉ is CH₂CH₂SO₃Na, and R₁₀ is ¹¹CH₃),

N-([¹¹C]methyl)deoxycholyltaurine (i.e. Formula 3 where R₁ and R₄ are α-OH, R₂ and R₃ are H, R₉ is CH₂CH₂SO₃Na, and R₁₀ is ¹¹CH₃),

N-([¹¹C]methyl)ursodeoxycholyltaurine (i.e. Formula 3 where R₁ is α-OH, R₃ is β-OH, R₂ and R₄ are H, R₉ is CH₂CH₂SO₃Na, and R₁₀ is ¹¹CH₃),

N-([¹¹C]methyl)lithocholyltaurine (i.e. Formula 3 where R₁ is α-OH, R₂, R₃ and R₄ are H, R₉ is CH₂CH₂SO₃Na, and R₁₀ is ¹¹CH₃).

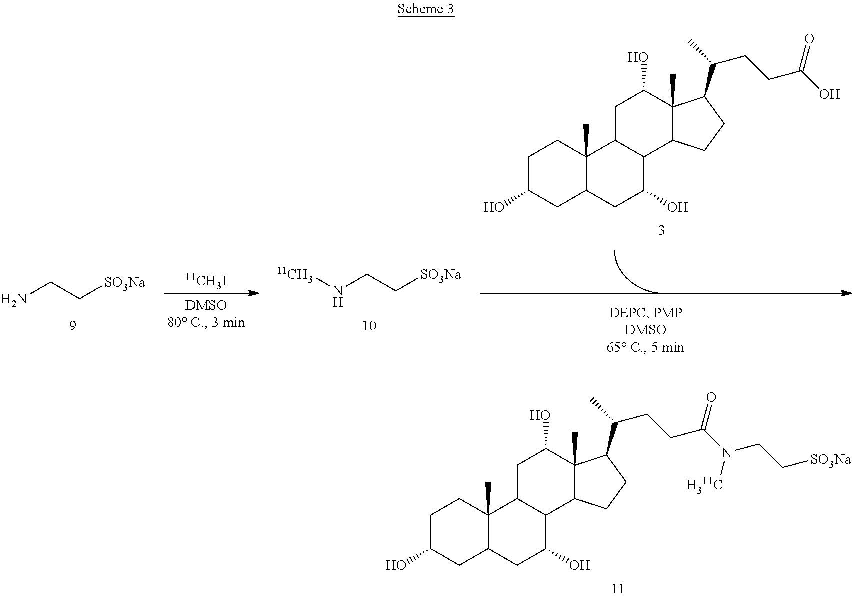

The radiosynthesis is performed using the GE Tracerlab™ FX_{C} Pro synthesis system: ¹¹CH₃I is delivered at room temperature to of the reactor containing taurine sodium (or potassium) salt 9 (8 μmol) in dry DMSO (300 μl). The reactor is heated in an oil bath at 80° C. for 3 min. Solutions of cholic acid 3 (29 μmol) and PMP (28 μmol; 5 μl) in dry DMSO (250 μl) and DEPC (29 μmol; 5 μl) in dry DMSO (150 μl) are then successively added. The reaction mixture is heated at 65° C. for 5 min and then quenched with 15% ethanol in water (0.7 ml; more ethanol is used for bile acids more lipophilic than cholic acid; up to 50%). The formed compound II is purified by preparative reverse phase HPLC on a Phenomenex® Synergi™ Hydro-RP (4 μm, 21.2×100 mm) column with 30% acetonitrile in sterile aqueous 70 mM NaH₂PO₄ (more acetonitrile is used for bile acids more lipophilic than cholic acid; up to 40%) as mobile phase (flow 15 ml/min; λ=220 nm). The fraction containing 11 is collected and diluted with water (50 ml), before passed slowly over a C₈ solid phase extraction cartridge (preconditioned with 10 ml ethanol, followed by 10 ml water) on which 11 is trapped. The cartridge is washed with water (15 ml) before 11 is eluted from the cartridge using ethanol (1 ml). The ethanolic solution is diluted with sterile 70 mM NaH₂PO₄ (9 ml) to give the final aqueous solution of product 11 (1.9-5.8 GBq) with a radiochemical purity of more than 99%. The radiochemical yield of the reaction is 75±2% (mean±SD; n=3) with a total synthesis time of 40 min. A similarly high amount (1.0-6.4 GBq), high radiochemical purity (>99%) and high radiochemical yield (70-80%) are obtained for other analogues such as the four stated above.

### Example 4

### Preparation of N-([¹⁸F]fluoromethyl)cholyltaurine (N-(3α,7α,12α-Trihydroxy-24-oxocholan-24-yl)-N—[¹⁸F]fluoromethyl-taurine)

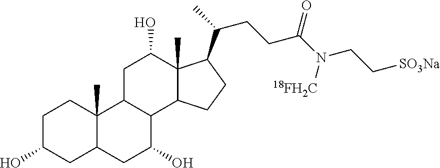

Radiosynthesis

The following radiosynthesis (Scheme 4) cover the compounds according to Formula 1 and Formula 3, wherein R₉ is not COOH and R₁₀ is CH₂¹⁸F.

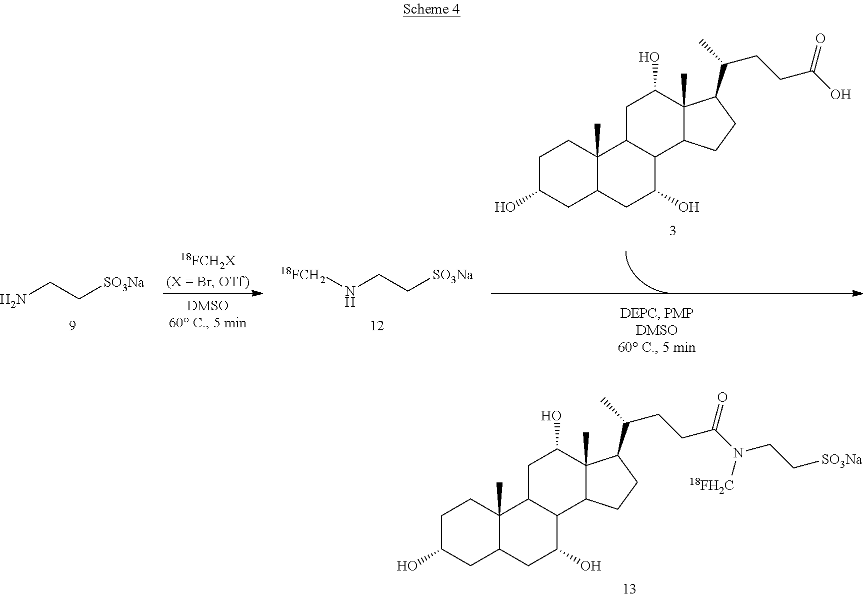

¹⁸FCH₂X (X=Br or OTf) is added to a mixture of taurine sodium (or potassium) salt 9 (8 μmol) in dry DMSO (300 μl) at room temperature. The sealed reaction vial is heated in an oil bath at 60° C. for 5 min. The vial is removed from the oil bath and solutions of cholic acid 3 (29 μmol) and PMP (28 μmol; 5 μl) in dry DMSO (250 μl) and DEPC (29 μmol; 5 μl) in dry DMSO (150 μl) are successively added. The reaction mixture is heated at 60° C. for 5 min and then quenched with water or aq. ethanol (4 ml). The formed compound 13 is purified by preparative HPLC using conditions determined by the stilled chemist. The fraction containing 13 is collected and diluted with water (50 ml), before passed slowly over a C₁₈ or C₈ solid phase extraction cartridge (preconditioned with 10 ml ethanol, followed by 10 ml water) on which 13 is trapped. The cartridge is washed with water (15 ml) before 13 is eluted from the cartridge using ethanol (1 ml). The ethanolic solution is diluted with sterile 70 mM NaH₂PO₄ (9 ml) to give the final aq. solution of product 13.

### Example 5

### Preparation of [24-¹¹C]cholic acid (3α,7α,12α-Trihydroxy-[24-¹¹C]cholan-24-oic acid)

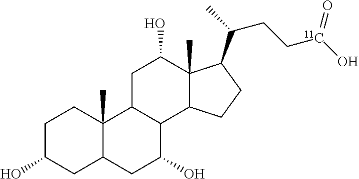 [SMILES: C[C@H](CC[11C](=O)O)[C@H]1CCC2C3C(C[C@H](O)[C@@]21C)[C@@]1(C)CC[C@@H](O)CC1C[C@H]3O]

The following radiosynthesis (Scheme 5) covers the compounds according to Formula 2.

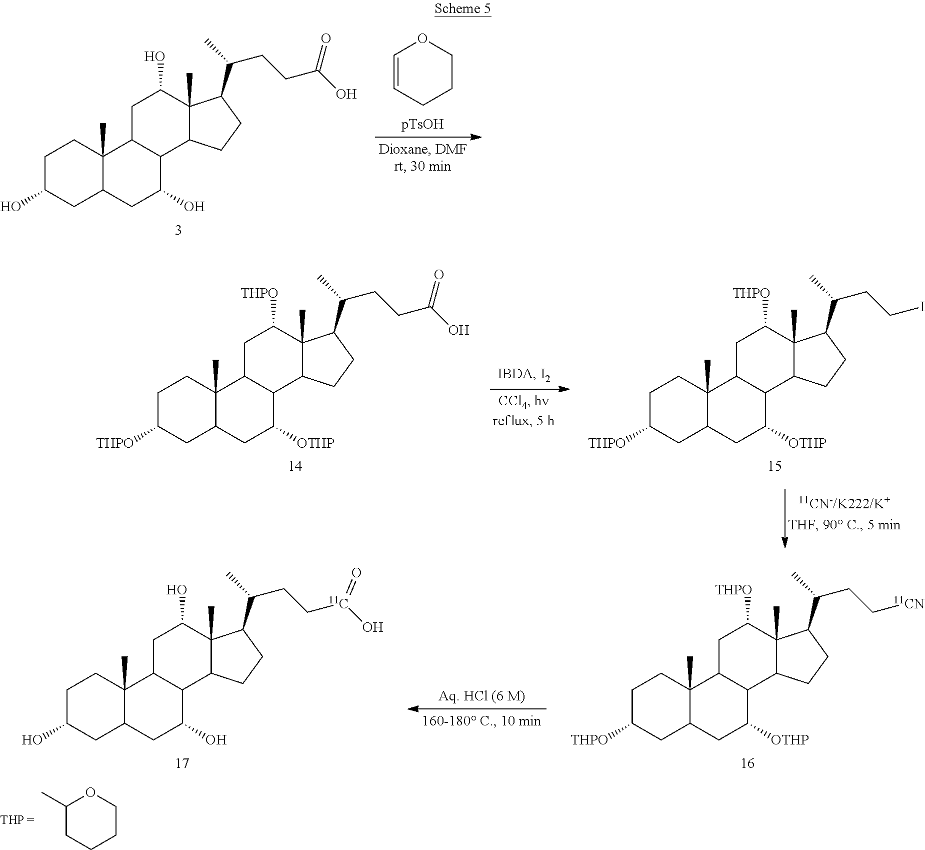

Compound 14 is prepared from cholic acid 3 as described in the literature (Rizzi et al. Org. Biomol. Chem., 2011, 9: 2899-2905). Compound 14 (0.66 g; 1.0 mmol) is then dissolved in CCl₄ (75 ml) and the mixture is irradiated (200 W tungsten lamp) at reflux temperature. Iodosobenzene diacetate, IBDA (3×177 mg; 3×0.55 mmol) and I₂ (3×127 mg; 0.50 mmol) is added in three portions with 1 h irradiation and reflux between each addition. After the third portions of IBDA and I₂ have been added, the mixture is irradiated at reflux temperature until the reaction is complete by TLC (ca. 3 h). The reaction mixture is cooled to room temperature, wash with saturated Na₂S₂O₃ (100 ml), then H₂O (100 ml). The organic solvent is removed by evaporation and the crude product 15 is purified by flash chromatography using conditions determined by the stilled chemist.

H¹¹CN is trapped in THF (300 μl) containing Kryptofix® 2.2.2, K222 (3 mg) and aqueous 5 M potassium hydroxide (1.5 μl). After trapping, a solution of compound 15 (1 mg) in THF (100 μl) is added and the mixture is heated at 90° C. for 5 min. Aqueous 6 M hydrochloric acid (1 ml) is then added and the temperature is increased to 160-180° C. After heating for 10 min, the reaction mixture is cooled to room temperature and neutralized with aq. NaOH. The crude product 17 is purified by preparative HPLC using conditions determined by the stilled chemist. The fraction containing 17 is collected and diluted with water (50 ml), before passed slowly over a C₁₈ or C₈ solid phase extraction cartridge (preconditioned with 10 ml ethanol, followed by 10 ml water) on which 17 is trapped. The cartridge is washed with water (15 ml) before 17 is eluted from the cartridge using ethanol (1 ml). The ethanolic solution is diluted with 70 mM NaH₂PO₄ (9 ml) to give the final aq. solution of product 17.

Derivatives

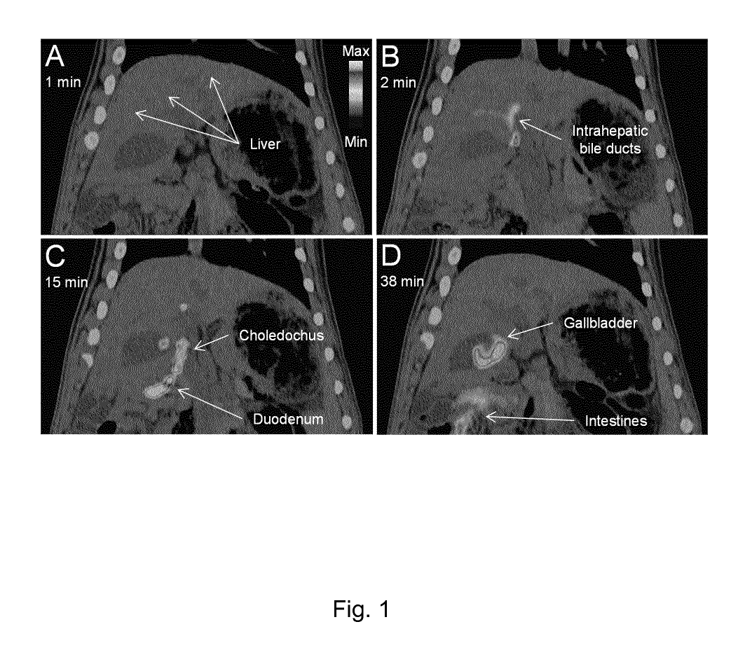

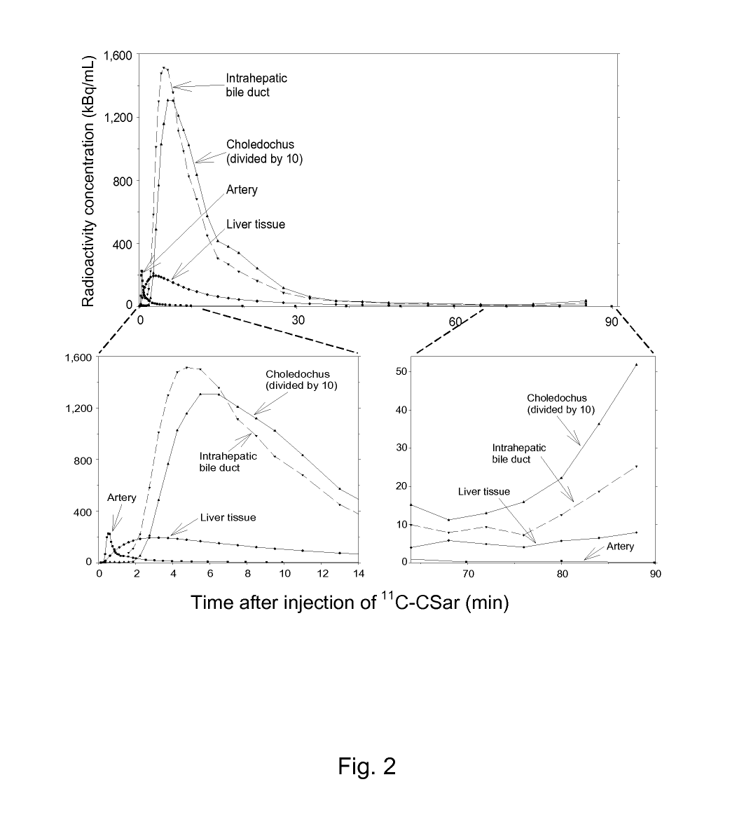

For the above procedures in examples 1 and 2, compound 1 may also be a methyl ester of one of the amino acids: Glycine, alanine, arginine, asparagine, asparaginic acid, cysteine, glutamine, glutamic acid, histidine, isoleucine, leucine, lysine, methionine, phenylalanine, serine, threonine, tryptophane, tyrosine, valine, or a salt hereof. Compounds, wherein R₉ is (CH₂)₁₋₄COOC₁₋₄alkyl (e.g. compounds 4 and 7, wherein R₉ is CH₂COOCH₃) are obtained by not performing the third reaction step (hydrolysis) illustrated in FIGS. 1 and 2.

For the above procedures in examples 3 and 4, compound 9 may have a structure according to claim 1 (e.g. NH₂—(CH₂)₁₋₄N⁺R₁₃R₁₄R₁₅, NH₂—(CH₂)₁₋₄OH, NH₂—(CH₂)₁₋₄O—C₁₋₈-alk(en/yn)yl). These compounds are prepared by a chemist stilled in the arts, if not commercially available.

In all of the above examples, compound 3 may have a structure according to claim 1 and one of the commercially available bile acids or bile acid analogues: 3α,7α-dihydroxy-5β-cholan-24-oic acid (chenodeoxycholic acid), 3α,12α-dihydroxy-5β-cholan-24-oic acid (deoxycholic acid), 3α,7β-dihydroxy-5β-cholan-24-oic acid (ursodeoxycholic acid), 3α-hydroxy-5β-cholan-24-oic acid (lithocholic acid), 3β-hydroxy-5β-cholan-24-oic acid (isolithocholic acid), 3α,7α,12α-trihydroxy-5α-cholan-24-oic acid (allocholic acid), 3,7,12-trioxo-5β-cholan-24-oic acid (dehydrocholic acid), 3α,6α,7α-trihydroxy-5β-cholan-24-oic acid (α-hyocholic acid), 3α,6α,7β-trihydroxy-5β-cholan-24-oic acid (β-hyocholic acid), 3α,6β,7β-trihydroxy-5β-cholan-24-oic acid (β-muricholic acid), 3α,7α,12α-trihydroxy-24-nor-5α-cholan-23-oic acid (allonorcholic acid), 3α,7α,12α-trihydroxy-24-nor-5β-cholanoic acid-(23) (24-norcholic acid), 3α,7α,12β-trihydroxy-5β-cholan-24-oic acid, 3β,7β,12α-trihydroxy-5β-cholan-24-oic acid, 3β,7β,12β-trihydroxy-5β-cholan-24-oic acid, 3α,7β,12α-trihydroxy-5β-cholan-24-oic acid, 3β,7α,12β-trihydroxy-5β-cholan-24-oic acid, 3β,7α,12α-trihydroxy-5β-cholan-24-oic acid, 3α,7β,12β-trihydroxy-5β-cholan-24-oic acid, 3α-hydroxy-5α-cholan-24-oic acid, 3β-hydroxy-5α-cholan-24-oic acid, 5β-cholan-24-oic acid, 5α-cholan-24-oic acid, (25S or 25R)-3α,7α-dihydroxy-5β-cholestan-26-oic acid, (25S or 25R)-3α,7α-dihydroxy-5α-cholestan-26-oic acid, (25S or 25R)-3β,7α-dihydroxy-5α-cholestan-26-oic acid, (25S or 25R)-3α,7α,12α-trihydroxy-5β-cholestan-26-oic acid, (25S or 25R)-3α,7α,12α-trihydroxy-5α-cholestan-26-oic acid or a salt hereof.

Compounds according to claim 1 not listed above, are prepared by a chemist stilled in the arts, if not commercially available.

### Example 6

### In Vivo Studies

In Vivo Studies in Pigs

Three pigs (3-month old female Danish Landrace and Yorkshire cross-breed; body weight 35-40 kg) were fasted for 18 h with free access to water. Each animal was premedicated with midazolam and s-ketamine, anaesthetized with a mixture of midazolam, s-ketamine and propofol, and mechanically ventilated as previously described (Sørensen M. Munk O L, Mortensen F V, et al. Hepatic uptake and metabolism of galactose can be quantified in vivo by 2-[¹⁸F]fluoro-2-deoxy-galactose positron emission tomography. Am J Physiol Gastrointest Liver Physiol. 2008; 295:G27-G36). The animal was placed on a thermostatically-controlled heating blanket, keeping the rectal temperature 38.5-39.5° C. By adjusting the mechanical respiration, arterial blood pCO₂, pO₂, and pH were kept between 5.3-7.2 kPa, 12-25 kPa, and 7.35-7.45, respectively. Blood glucose was 5.0-6.7 mM. Catheters (Cordis) were inserted into the femoral vein and artery for intravenous administrations and blood sampling, respectively. After completion of the experiment, the animal was euthanized with an overdose of pentobarbital (100 mg/kg).

The studies were performed according to the Danish Animal Experimentation Act and the European convention for the protection of vertebrate animals used for experimental and other purposes (ETS No. 123).

For PET/CT studies, the pig was placed on the back in a Siemens Biograph 64 Truepoint PET/CT camera with a 21 cm transaxial PET field-of-view. A CT scan (250 effective mAs with CAREDose4D, 120 kV, pitch 1.0, slice thickness 2.0 mm) was performed for definition of anatomical structures and attenuation correction of the PET recordings.

Pigs 1 and 2 underwent 90-min dynamic PET/CT recordings (list-mode) using intravenous administration of 400 MBq ¹¹C—CSar. In Pig 1, a second dynamic ¹¹C—CSar PET/CT was preceded by intravenous 50-second infusion of 286 mg/kg pig cholyltaurine to investigate possible inhibition of the transhepatic transport of ¹¹C—CSar. In Pig 2, a second ¹¹C—CSar administration was given to determine the biodistribution of the tracer by means of whole-body PET/CT recordings. In both pigs, at least 6 half-lives of the ¹¹C-isotope (half-live: 20.4 min) were allowed to pass between tracer administrations.

The decay-corrected dynamic PET data were reconstructed using the iterative TrueX algorithm (3 iterations, 21 subsets) and post-filtered using a 3 mm Gaussian filter yielding 3D-images of 168×168×109 voxels. Time-activity curves (TACs) were generated from volumes of interest (VOIs) drawn in liver tissue, intrahepatic bile ducts, and choledochus using fused PET/CT images.

During the dynamic PET recordings of Pig 1 and 2, blood samples (0.5 mL) were collected from the femoral artery at 12×5, 6×10, 6×30, 5×60, 8×600 seconds. The radioactivity concentrations in the samples were measured in a well counter (Packard Biosciences) that was cross-calibrated to the PET camera. Additional blood samples were collected 2, 5, 10, 20, 40, 60, and 90 min after tracer administration for determination of ¹¹C-metabolites in plasma.

Pig 3 did not undergo PET/CT, but was used to collect bile samples 15, 30, and 90 min after administration of ¹¹C—CSar for analysis of possible ¹¹C-metabolitesin bile. The plasma and bile samples were fractionated by HPLC (monitored by serial UV detection (λ=220 nm) and radiodetection) and radioactivity concentrations were measured in the well counter. Two different HPLC conditions were used: Sphereclone™ SAX (Phenomenex®, 250×4.6 mm) using 95% methanol and 5% acetic acid, pH 4, as eluent; or, Sphereclone™ ODS(2) C-18 (Phenomenex®, 250×4.6 mm) with a mixture of acetonitrile and aqueous 70 mM Na₂HPO₄ (60:40) as eluent.

The plasma free fraction of ¹¹C—CSar was determined in plasma samples collected prior to tracer administration in all three pigs; moreover, plasma was collected 10 seconds after end of cholyltaurine infusion in Pig 1, i.e. before the ¹¹C—CSar administration. The plasma samples were mixed with aliquots of ¹¹C—CSar, pipetted into ultrafiltration units (Pall Nanosep Centrifugal Device, Cut-off 30,000 D, Sigma-Aldrich) and centrifuged at room temperature (10 min at 12,000 rpm). Radioactivity in plasma and ultrafiltrates were counted in the well counter. Filter retention of the tracer was determined using sodium phosphate buffer solution (35 mM; pH 7.2). The free fraction was calculated as the ratio of radioactivity concentration in the ultrafiltrate corrected for filter retention to the total radioactivity concentration in plasma.

For the biodistribution study, ¹¹C—CSar was administrated intravenously in Pig 2 followed by five whole-body PET scans with one minute between scans and with progressive increase of scan durations per bed-position, i.e. 1, 2, 3, 4 and 5 min, respectively. Organs with high accumulation of tracer relative to surrounding tissue were identified by visual inspection (liver, gallbladder, stomach, and small intestines) and were included as individual source organs. Virtually no radioactivity was detected in other organs, including the urinary bladder. The liver had a uniform radioactivity distribution and accordingly the total liver radioactivity was estimated as the radioactivity concentration in a central VOI multiplied by the liver volume. Other source organs (gallbladder, stomach, small intestines, and kidneys) had non-uniform radioactivity distribution and for these organs, the total activity was estimated using a large VOI encompassing all accumulated radioactivity. For each source organ, the time course of the non-decay-corrected total radioactivity was generated. Data were extrapolated from pig (40 kg) to human (74 kg) and recalculated into time courses of fractions of injected dose (% ID). Residence times were computed as the trapezoidal sum of the time course of % ID assuming that the radioactivity decayed only by physical decay after the last scan. Residence time for the rest of the body was calculated as the total body residence time (without voiding) minus the sum of the residence times from the source organs. The residence times were entered into OLINDA/EXM 1.0 (Stabin M G, Sparks R B, Crowe E. OLINDA/EXM: The second-generation personal computer software for internal dose assessment in nuclear medicine. J Nucl Med. 2005; 46:1023-1027) to compute absorbed doses using the male reference phantom and to obtain effective dose values according to ICRP 60.

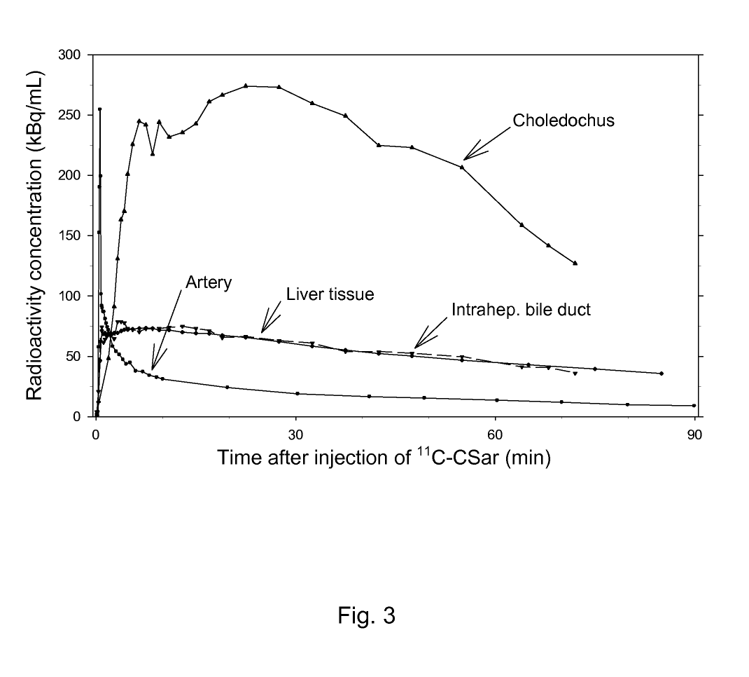

The dynamic PET/CT recordings in Pig 1 and 2 showed a rapid uptake of ¹¹C—CSar into liver tissue (FIG. 1A) with subsequent excretion into the intrahepatic bile ducts and the common hepatic bile duct (FIG. 1B). From here, most of the tracer was excreted via the choledochus into duodenum, while the remainder of the tracer entered the gallbladder (FIG. 1C). Eventually, the tracer was dispersed into the intestines or concentrated in the gallbladder (FIG. 1D). FIG. 3 shows TACs for these structures. The liver TAC peaked within 3 minutes, with a radioactivity concentration comparable to the peak concentration of the arterial TAC, and then decreased rapidly to very low concentrations within 30-40 min. Radioactivity appeared in the intrahepatic bile ducts approximately one min after ¹¹C—CSar administration and in the choledochus one minute later. The radioactivity concentration in the choledochus peaked 4-6 min after tracer administration and the TACs illustrate how ¹¹C—CSar was concentrated in the biliary tree (FIG. 2). As a result of enterohepatic circulation, radioactivity concentrations in the liver tissue and bile ducts increased again about 75 min after administration of the tracer. Analysis of plasma from Pigs 1 and 2, and bile samples from Pig 3 showed no ¹¹C-metabolites up to 90 min after ¹¹C—CSar administration, verifying that ¹¹C—CSar does not undergo hepatic or intestinal metabolism. The free fraction of ¹¹C—CSar added to plasma samples collected before tracer administration was 18±2% (n=3) which is similar to that reported for endogenous cholyl-conjugates, i.e. 20-40%.

Pretreatment with cholyltaurine before the second scan of Pig 1 markedly inhibited hepatic uptake and biliary excretion of ¹¹C—CSar as illustrated by the TACs in FIG. 3. Compared to the TACs from the PET/CT studies without cholyltaurine, the liver tissue TACs only reached a maximum radioactivity concentration of approximately 40% and the subsequent decrease with time was significantly slower. Moreover, the intrahepatic bile ducts were indistinguishable from surrounding liver tissue and the TAC in the choledochus increased at a much slower rate and reached a maximum value that was only 2% of that without pretreatment with cholyltaurine (FIG. 2 vs. FIG. 3). These findings are in accordance with the hypothesis that ¹¹C—CSar and cholyltaurine compete for one or more of the same transporters from blood to liver cells and from hepatocytes to bile capillaries. The free fraction of ¹¹C—CSar in plasma samples collected after administration of cholyltaurine was 100%, showing competition for protein binding between tracer and cholyltaurine.

The estimated radiation doses were highest in the gallbladder wall, small intestines, liver, and upper large intestine wall (Table 2). The effective absorbed dose of ¹¹C—CSar was 4.4 μSv/MBq.

TABLE 2
Absorbed Dose Estimates of ¹¹C-CSar*

| Target organ | μGy/MBq |
|---|---|
| Adrenals | 2.88 |
| Brain | 1.57 |
| Breasts | 1.66 |
| Gallbladder wall | 59.40 |
| Lower large intestine wall | 3.91 |
| Small intestine | 39.30 |
| Stomach wall | 3.92 |
| Upper large intestine wall | 7.07 |
| Heart wall | 2.33 |
| Kidneys | 2.34 |
| Liver | 10.80 |
| Lungs | 2.06 |
| Muscle | 2.23 |
| Ovaries | 4.97 |
| Pancreas | 3.36 |
| Red marrow | 2.34 |
| Bone surface | 2.87 |
| Skin | 1.61 |
| Spleen | 2.37 |
| Testes | 1.82 |
| Thymus | 1.88 |
| Thyroid | 1.78 |
| Urinary bladder wall | 2.67 |
| Uterus | 4.62 |
| Total body | 2.83 |
| Effective dose (μSv/MBq) | 4.40 |

*The data was obtained from ¹¹C-CSar PET/CT biodistribution study in Pig 2 (40 kg), being extrapolated to 74-kg human data.

In Vivo Studies in Humans

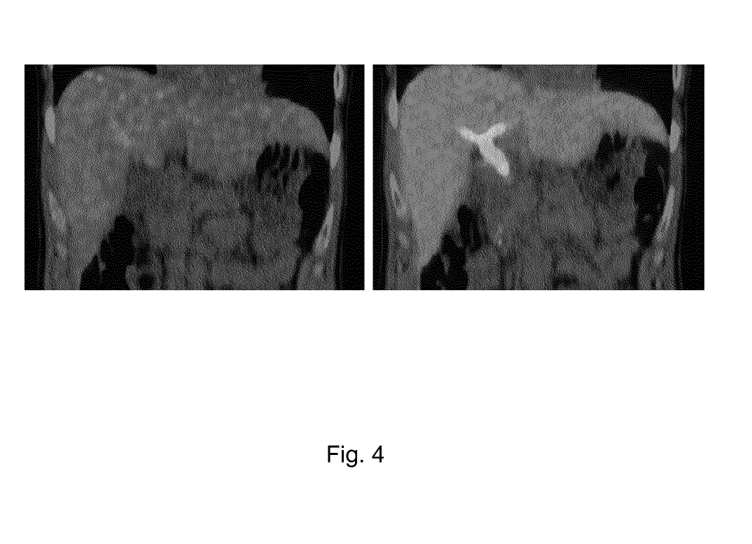

Two dynamic PET/CT studies in humans have been performed; one in a patient with inherited cholestasis and the other in a patient with drug-induced cholestasis. FIG. 4 illustrates the findings in the patient with inherited cholestasis; a 45-year old man diagnosed with BRIC-1 (benign recurrent intrahepatic cholestasis), who was examined during a cholestatic episode with severe pruritus, hyperbilirubinemia and high plasma bile acid concentrations. The study clearly showed that both the uptake of bile acids from blood to hepatocytes as well as excretion from hepatocytes to bile was impaired in this patient. After 5 min, ¹¹C—CSar was still observed in the liver in contrast to healthy persons where it passes through the liver within a few minutes. After 25 min, ¹¹C—CSar accumulated in the liver and intrahepatic bile ducts, which is in contrast to healthy persons where ¹¹C—CSar is no longer observable in the hepato-biliary system at this time point. The study clearly shows that both the uptake of bile acids from blood to hepatocytes as well as excretion from hepatocytes to bile was impaired in this patient. Images from the second patient show similar changes.

### Example 7

### Preparation of N-(2-[¹⁸F]fluoroethyl)cholylglycine (N-(3α,7α,12α-trihydroxy-24-oxocholan-24-yl)-N-(2-[¹⁸F]fluoroethyl-glycine)

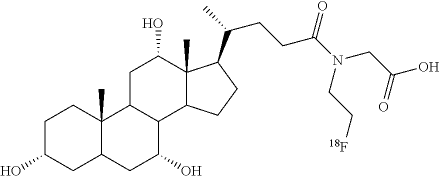 [SMILES: C[C@H](CCC(=O)N(CC[18F])CC(=O)O)[C@H]1CCC2C3C(C[C@H](O)[C@@]21C)[C@@]1(C)CC[C@@H](O)CC1C[C@H]3O]

Radiosynthesis

The following radiosynthesis (Scheme 6) cover the compounds according to Formula 1 and Formula 3, wherein R₉ is a COOH group and R₁₀ is (CH₂)₂¹⁸F. A similar method of preparation is used when R₁₀ is (CH₂)₃₋₈¹⁸F (the method used when R₁₀ is CH₂¹⁸F, is described in example 2).

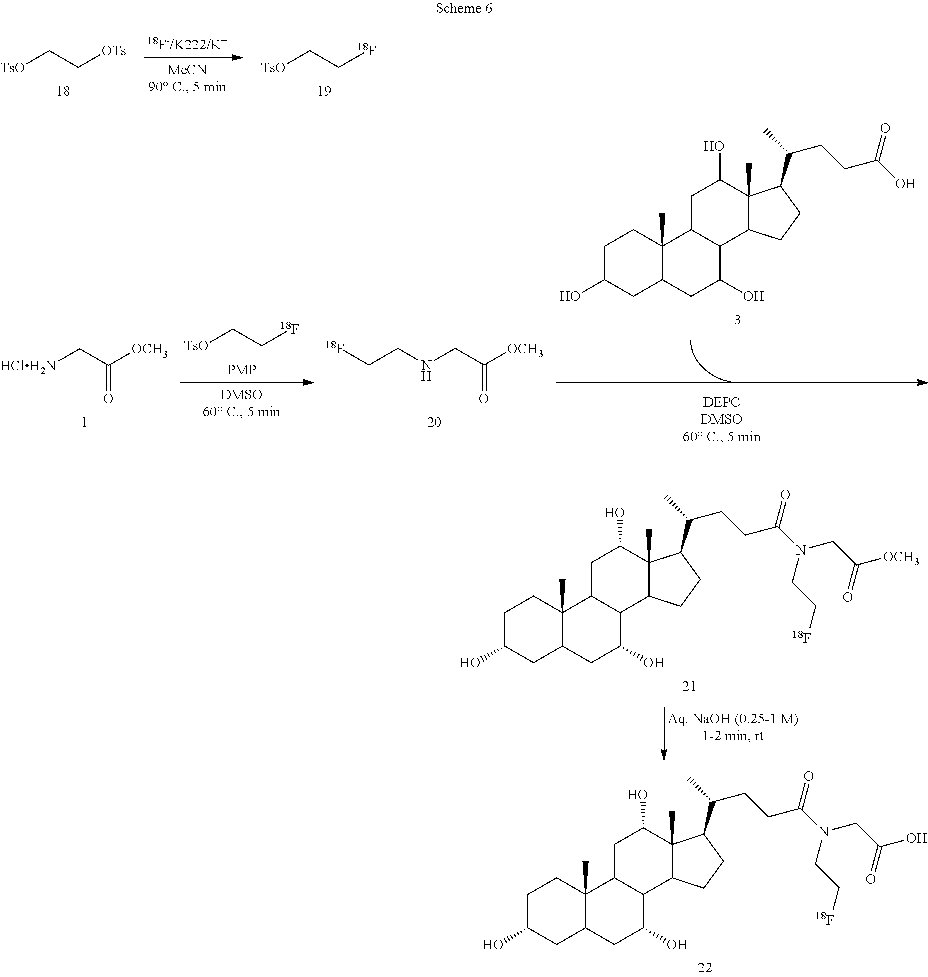

The complex ¹⁸F/K222/K⁺ in acetonitrile, prepared as previously described, is added a solution of ethylene di(p-toluenesulfonate) 18 (5 mg; 14 μmol) in dry acetonitrile (total volume approx. 0.5 ml) and the mixture is heated at 90° C. for 5 min to give [¹⁸F]fluoro tosylate 19. The reaction mixture is then evaporated to dryness. To the dry [¹⁸F]fluoro tosylate 19 is added a solution of glycine methyl ester hydrochloride 1 (0.8 mg; 6 μmol) and PMP (5 ml; 28 μmol) in dry DMSO (300 μl) and the sealed reaction vial is heated in an oil bath at 60° C. for 5 min. The reaction vial is removed from the oil bath and solutions of cholic acid 3 (29 μmol) in dry DMSO (250 μl) and DEPC (29 μmol; 5 μl) in dry DMSO (150 μl) are successively added. The reaction mixture is heated at 60° C. for 5 min and then quenched with water or aq. ethanol (4 ml). The formed compound 21 is purified by preparative HPLC using conditions determined by the stilled chemist. The fraction containing 21 is collected and diluted with water (50 ml), before passed slowly over a C₁₈ or C₈ solid phase extraction cartridge (preconditioned with 10 ml ethanol, followed by 10 ml water) on which 21 is trapped. The cartridge is washed with water (15 ml) before 21 is eluted from the cartridge using ethanol (1 ml). Aqueous 0.25-1 M NaOH (2 ml) is then passed through the cartridge into the ethanolic solution. The alkaline mixture is allowed to stand for 1-2 min at room temperature, before finally neutralized with aq. NaH₂PO₄ or citrate buffer (7 ml) to give a neutral aq. solution of the final product 22.

### Example 8

### Preparation of N-(2-[¹⁸F]fluoroethyl)cholyltaurine (N-(3α,7α,12α-Trihydroxy-24-oxocholan-24-yl)-N-(2-[¹⁸F]fluoroethyl-taurine)

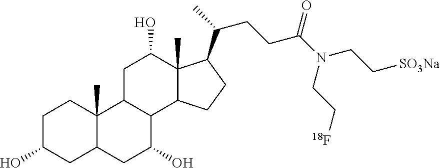

The following radiosynthesis (Scheme 7) cover the compounds according to Formula 1 and Formula 3, wherein R₉ is not COOH and R₁₀ is (CH₂)₂¹⁸F. A similar method of preparation is used when R₁₀ is (CH₂)₃₋₈¹⁸F (the method used when R₁₀ is CH₂¹⁸F, is described in example 4).

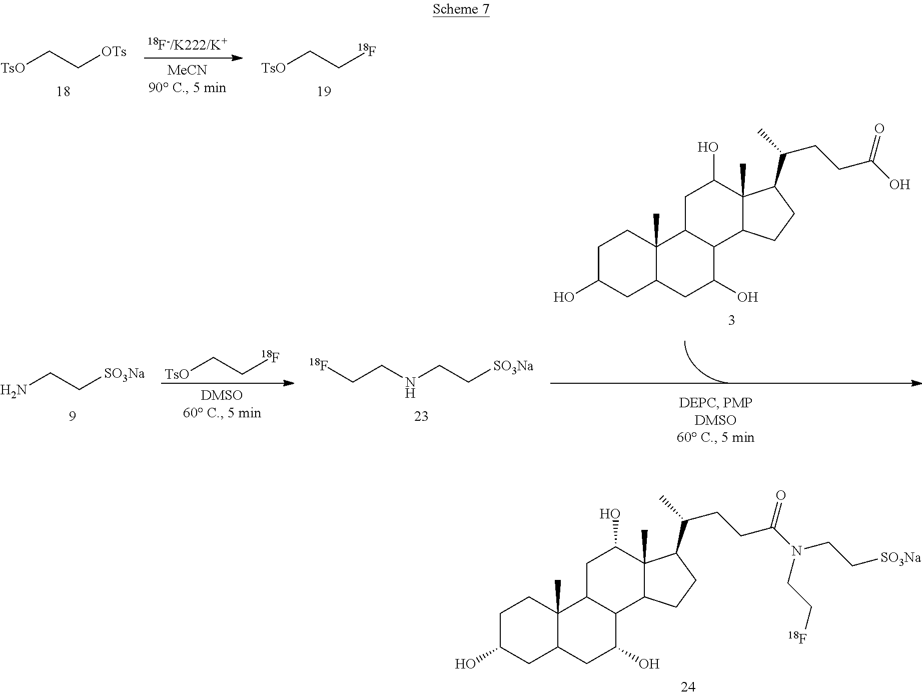

The complex ¹⁸F⁻/K222/K⁺ in acetonitrile, prepared as previously described, is added a solution of ethylene di(p-toluenesulfonate) 18 (5 mg; 14 μmol) in dry acetonitrile (total volume approx. 0.5 ml) and the mixture is heated at 90° C. for 5 min to give [¹⁸F]fluoro tosylate 19. The reaction mixture is then evaporated to dryness. To the dry [¹⁸F]fluoro tosylate 19 is added a solution of taurine sodium (or potassium) salt 9 (8 μmol) in dry DMSO (300 μl) at room temperature. The sealed reaction vial is heated in an oil bath at 60° C. for 5 min. The vial is removed from the oil bath and solutions of cholic acid 3 (29 μmol) and PMP (28 μmol; 5 μl) in dry DMSO (250 μl) and DEPC (29 μmol; 5 μl) in dry DMSO (150 μl) are successively added. The reaction is heated at 60° C. for 5 min then quenched with water or aq. ethanol (4 ml). The formed compound 24 is purified by preparative HPLC using conditions determined by the stilled chemist. The fraction containing 24 is collected and diluted with water (50 ml), before passed slowly over a C₁₈ or C₈ solid phase extraction cartridge (preconditioned with 10 ml ethanol, followed by 10 ml water) on which 24 is trapped. The cartridge is washed with water (15 ml) before 24 is eluted from the cartridge using ethanol (1 ml). The ethanolic solution is diluted with sterile 70 mM NaH₂PO₄ (9 ml) to give the final aq. solution of product 24.

1. A radiolabeled compound comprising the structure of Formula 1: 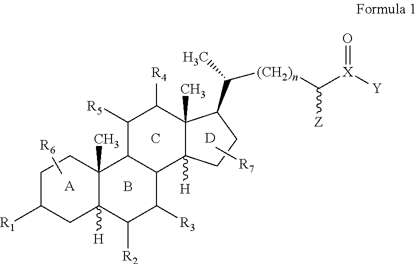
or a salt and/or hydrate thereof;
wherein:
said compound comprises a steroid structure (ABCD) and at least one radioactive isotope selected from the group consisting of ¹¹C and ¹⁸F
n is 0, 1, 2 or 3
X is C or ¹¹C
Z is H or —CH₃
Y is selected from the group consisting of OH, OR₈, C₁₋₆-alk(en/yn)yl, NR₉R₁₀
R₁, R₂, R₃, R₄, R₅, R₆ and R₇ are individually selected from the group consisting of H, OH, C₁₋₆-alk(en/yn)yl, aryl, halo-C₁₋₆-alk(en/yn)yl, C₃₋₈-cycloalk(en)yl, halo-C₃₋₈-cycloalk(en)yl, and hydroxy-C₁₋₆-alk(en/yn)yl, cyano, halogen, oxo, OSO₂OH, —CF₃, and NR₁₁R₁₂
R₈ is selected from the group consisting of C₁₋₆-alk(en/yn)yl, aryl and O₃₋₈-cycloalk(en)yl, halo-C₁₋₆-alk(en/yn)yl, halo-C₃₋₈-cycloalk(en)yl
R₉ is selected from the group consisting of H, C₁₋₆-alk(en/yn)yl, —COOH, —CHO, —CH₂COOH, —CH₂COOC₁₋₄alkyl, —CH₂SO₂OH, —CH₂CH₂COOH, —CH₂CH₂COOC₁₋₄alkyl, —CH₂CH₂SO₂OH, —(CH₂)₁₋₄N⁺R₁₃R₁₄R₁₅, —CH(CH₃)COOH, —CH((CH₂)₃NHC(NH)NH₂)COOH, —CH(CH₂CONH₂)COOH, CH(CH₂COOH)COOH, —CH(CH₂SH)COOH, —CH(CH₂CH₂CONH₂)COOH, —CH(CH₂CH₂COOH)COOH, —CH(CH₂C₃N₂H₃)COOH, —CH(CH(CH₃)CH₂CH₃)COOH, —CH((CH₂)₄NH₂)COOH, —CH((CH₂)₂SCH₃)COOH, —CH(CH₂C₆H₅)COOH, —CH(CH₂OH)COOH, —CH(CH(CH₃)OH)COOH, —CH(CH₂C₈H₆)COOH, —CH(CH₂C₆H₄OH)COOH, —CH(CH(CH₃)₂)COOH
R₁₀ is selected from the group consisting of H, C₁₋₈-alk(en/yn)yl, ¹¹CH₃, —(CH₂)₁₋₈F, —(CH₂)₁₋₈¹⁸F, CH₂—C₃₋₈-cycloalk(en)yl and CH₂-halo-C₃₋₈-cycloalk(en)yl
R₁₁ and R₁₂ are individually selected from the group consisting of H, C₁₋₆-alk(en/yn)yl, ¹¹CH₃, aryl, C₃₋₈-cycloalk(en)yl
R₁₃, R₁₄ and R₁₅ are individually selected from the group consisting of C₁₋₈-alk(en/yn)yl.

2. The compound according to claim 1, wherein said steroid structure (ABCD) comprises one or more double bonds.

3. The compound according to any of claims 1 and 2, wherein R₁, R₃ and R₄ is H or OH.

4. The compound according to claim 3, wherein R₁ is OH.

5. The compound according to claim 4, wherein R₁, R₃ and R₄ is OH.

6. The compound according to claim 5, wherein R₁, R₃ and R₄ is OH in an α-position.

7. The compound according to any of the preceding claims, wherein n=1.

8. The compound according to any of the preceding claims, wherein R₂, R₅, R₆ and R₇ is H.

9. The compound according to any of the preceding claims, wherein Y is OH.

10. The compound according to any of the preceding claims, wherein Y is NR₉R₁₀.

11. The compound according to claim 10, wherein R₉ is CH₂COOH.

12. The compound according to claim 1 comprising the structure of Formula 2: 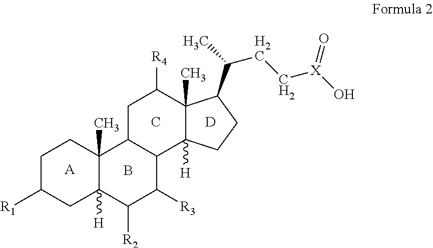
or a salt and/or hydrate thereof;
wherein:
X is ¹¹C
R₁, R₂, R₃ and R₄ are individually selected from the group consisting of H, OH, C₁₋₆-alk(en/yn)yl, aryl, halo-C₁₋₆-alk(en/yn)yl, C₃₋₈-cycloalk(en)yl, halo-C₃₋₈-cycloalk(en)yl, and hydroxy-C₁₋₆-alk(en/yn)yl, cyano, halogen, oxo, OSO₂OH, —CF₃, and NR₁₁R₁₂
R₁₁ and R₁₂ are individually selected from the group consisting of H, C₁₋₆-alk(en/yn)yl, aryl, C₃₋₈-cycloalk(en)yl.

13. The compound according to claim 12, wherein R₁ is OH.

14. The compound according to claim 12, wherein R₁ and R₄ are OH.

15. The compound according to claim 12, wherein R₁, R₃ and R₄ are OH and R₂ is H.

16. The compound according to claim 1 comprising the structure of Formula 3: 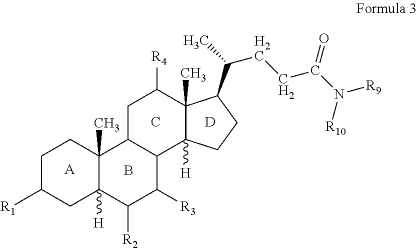
or a salt and/or hydrate thereof;
wherein:
R₁, R₂, R₃, and R₄ are individually selected from the group consisting of H, OH, C₁₋₆-alk(en/yn)yl, aryl, halo-C₁₋₆-alk(en/yn)yl, —C₃₋₈-cycloalk(en)yl, halo-C₃₋₈-cycloalk(en)yl, and hydroxy-C₁₋₆-alk(en/yn)yl, cyano, halogen, oxo, OSO₂OH, —CF₃, and NR₁₁R₁₂
R₉ is selected from the group consisting of H, —C₁₋₆-alk(en/yn)yl, —COOH, —CHO, —CH₂COOH, —CH₂COOC₁₋₄alkyl, —CH₂SO₂OH, —CH₂CH₂COOH, —CH₂CH₂COOC₁₋₄alkyl, —CH₂CH₂SO₂OH, —(CH₂)₁₋₄N⁺R₁₃R₁₄R₁₅
R₁₀ is selected from the group consisting of H, —C₁₋₈-alk(en/yn)yl, —¹¹CH₃, —(CH₂)₁₋₈F, —(CH₂)₁₋₈¹⁸F, —CH₂—C₃₋₈-cycloalk(en)yl, —CH₂-halo-C₃₋₈-cycloalk(en)yl
R₁₁ and R₁₂ are individually selected from the group consisting of H, O₁₋₆-alk(en/yn)yl, aryl, C₃₋₈-cycloalk(en)yl
R₁₃, R₁₄ and R₁₅ are individually selected from the group consisting of —C₁₋₈-alk(en/yn)yl.

17. The compound according to claim 16, wherein R₁, R₃ and R₄ are OH and R₂ is H.

18. The compound according to claim 17, wherein R₁, R₃ and R₄ are OH in α-position.

19. The compound according to any of claim 16-18, wherein R₉ is —CH₂COOH and R₁₀ is ¹¹CH₃.

20. The compound according to any of claim 16-18, wherein R₉ is —CH₂COOH and R₁₀ is CH₂¹⁸F.

21. The compound according to any of claim 16-18, wherein R₉ is —CH₂CH₂SO₂OH and R₁₀ is ¹¹CH₃.

22. The compound according to claim any of claim 16-18, wherein R₉ is —CH₂CH₂SO₂OH and R₁₀ is CH₂¹⁸F.

23. The compound according to any of the preceding claims wherein H at position 5 is in β-position and H at position 14 is in α-position.

24. The compound according to any of the preceding claims wherein said compound is a bile acid.

25. A compound according to any one of claims 1 to 24 for use in an imaging method.

26. The compound according to claim 25, wherein the imaging method is planar scintigraphy.

27. The compound according to claim 25, wherein the imaging method is single-photon emission computed tomography (SPECT).

28. The compound according to claim 25, wherein the imaging method is positron emission tomography (PET).

29. The compound according to any of claims 27-28, wherein the imaging method is coupled to computed tomography (CT) or magnetic resonance imaging (MRI).

30. An imaging method comprising:
providing a compound according to any one of claims 1 to 29
administering said compound to an individual
making a radiographic image of a region of interest from said individual

31. The imaging method according to claim 30, wherein said radiographic image is obtained by planar scintigraphy.

32. The imaging method according to claim 30, wherein said radiographic image is obtained by SPECT.

33. The imaging method according to claim 30, wherein said radiographic image is obtained by PET.

34. The imaging method according to any of claims 32-33, wherein said radiographic image is obtained by SPECT or PET coupled to CT or MRI.

35. A method for diagnosing a disease in an individual said method comprising:
providing a compound according to any one of claims 1 to 29
administering said compound to said individual
making a radiographic image of at least a part of the body from said individual.

36. A method for determining the biliary excretory function in an individual said method comprising:
providing a compound according to any one of claims 1 to 29
administering said compound to said individual
making a radiographic image of at least a part of the body from said individual.

37. A method for evaluating the course of disease in an individual said method comprising:
providing a compound according to any one of claims 1 to 29
administering said compound to said individual
making a radiographic image of at least a part of the body from said individual.

38. A method for evaluating the effect of treatment of a disease in an individual, said method comprising:
providing a compound according to any one of claims 1 to 29
administering said compound to said individual
making a radiographic image of at least a part of the body from said individual.

39. The method according to claim 38, wherein a first radiographic image is made at a time point x and comparing said first radiographic image with a second radiographic image obtained from said individual at another time point y.

40. The method according to claim 39, wherein the time point x is before initiating the treatment of said individual and the time point y is after initiating the treatment of said individual.

41. The method according to claim 38, wherein a first radiographic image is taken at a first time point x during treatment of said individual and compared with a second radiographic image obtained form said individual and wherein said second radiographic image is taken at a second time y point during treatment of said individual.

42. The method according to any of claims 35-41, wherein said radiographic image is obtained by planar scintigraphy.

43. The method according to any of claims 35-41, wherein said radiographic image is obtained by SPECT.

44. The method according to any of claims 35-41, wherein said radiographic image is obtained by PET.

45. The method according to any of claims 43-44, wherein said radiographic image is obtained by SPECT or PET coupled to CT or MRI.

46. The method according to any of claims 35-45, wherein said disease is a hepatic, biliary and/or a gastro-intestinal disorder.

47. The method according to claim 46, wherein said gastro-intestinal disorder is a disorder in the small intestine.

48. The method according to claim 46, wherein said hepatic disorder is hepatic cancer or a cholestatic disorder.

49. The method according to any of claims 35-48, wherein said at least a part of the body is the gastro-intestinal region.

50. The method according to any of claims 35-48, wherein said at least a part of the body is at least a part of the hepato-biliary system.

51. The method according to claim 50, wherein said at least a part of the hepato-biliary system is the liver.

52. The method according to any of claims 35-51, wherein the compound is administered in a dosage is 3-6 MBq per kilo body weight.
